(19) United States
(12) Patent Application Publication
(10) Pub. No.: US 2006/0121483 A1
(43) Pub. Date: Jun. 8, 2006

(54) Methods for detecting teneurin signalling and related screening methods

(76) Inventors: Claudia Bagutti, Riehen (CH); Ruth Chiquet-Ehrishmann, Pratteln (CH); Krysztof Piotr Drabikoswki, Freiburg (DE); Beatrix Paulette Rubin-Lucht, Basel (CH)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/530,542

(22) PCT Filed: Oct. 14, 2003
(86) PCT No.: PCT/EP03/11382

§ 371 Date: Oct. 27, 2005

(30) Foreign Application Priority Data
Oct. 15, 2002 (GB) 0223984.6

(51) Int. Cl. C12Q 1/68; G01N 33/574

(52) U.S. Cl. 435/6; 435/7.23

(57) ABSTRACT

Methods are provided for detecting teneurin signaling by determining the presence and/or amount of a cleaved teneurin product, and correlating the presence and/or amount of the cleaved teneurin product with teneurin signalling. The methods can be used for screening possible anti-tumour agents or agents that modulate neuronal differentiation. Test substances are assayed for their ability to increase or decrease the presence of the cytoplasmic domain of teneurin or of its respective targets, such as ponsin, PML an zic.

### FIELD OF THE INVENTION

The present invention relates to methods of detecting teneurin signalling and methods for screening agents effective in modulating teneurin signalling with applications In the fields of neurobiology and oncology. In particular, agents affecting teneurin signalling may be useful as anti-tumour and/or anti-tumourigenic agents, or for modulating neuronal differentiation, development or regeneration.

### BACKGROUND OF THE INVENTION

Teneurins are a family of type II transmembrane proteins originally discovered in Drosophila. The first member was Ten-a (Baumgartner and Chiquet-Ehrismann (1993) Mech Dev 40:165-76; Minet and Chiquet-Ehrismann (2000) Gene 257: 87-97), which was found in a search for Drosophila homologues of tenascins and shares with this protein family the same type of EGF-like repeats. The second member of the teneurin family, Drosophila Ten-m/Odd oz (Odz; Baumgartner et al. (1994) Embo J 13: 3728-40) is expressed in seven stripes during the blastoderm stage of early embryos. Mutational analysis showed that ten-n/odz is a member of the “pair-rule” gene family and encodes for an extracellular protein having a central role in determining the segmentation of the embryo. The expression pattern in the developing embryos and the adult fly suggest further important activities of this protein in later developmental processes since its presence often coincides with locations of morphogenetic cell movements, in particular, during gastrulation, the development of the tracheal system, on pioneering axons and in the developing eye (Baumgartner et al. (1994) Embo J 13: 3728-40); (Levine et al. (1994) Cell 77: 587-98); (Levine et al. (1997) Dev Dyn 209: 1-14).

Vertebrates contain four ten-a/ten-m homologues, termed ten-m1-4 (Oohashi et al. (1999) J Cell Biol 145: 563-77), odz1-4 (Ben-Zur et al. (2000) Dev Biol 217: 107-20), or teneurin 1-4 (Minet and Chiquet-Ehrismann (2000) Gene 257: 87-97), respectively. A recent study presented two new members of this family of proteins in chicken, namely, teneurin-1 and teneurin-2. Both the Drosophila and chicken proteins are expressed In early stages of embryonic development, In particular, in the developing nervous system and suggest an interaction or teneurin-2 with the cytoskeleton. Expression of recombinant teneurin-2 in a neuroblastoma cell line appears to lead to filopodia formation and enlarged growth cones (Rubin et al. (1999) Dev Biol 216: 195-209).

Mouse DOC4, the first vertebrate member of the teneurin family, was identified in a screen for proteins that were expressed in response to perturbation of protein folding in the endoplasmic reticulum (Wang et al. (1998) Embo J 17 3619-30). Mouse DOC4 and all other vertebrate teneurins identified—namely, the mouse teneurins ten-m1-4/odz1-4 (Oohashi et al. (1999) J Cell Biol 145: 563-7);(Ben-Zur et al. (2000) Dev Biol 217: 107-20), the rat teneurin-2 orthologue, neurestin (Otaki and Firestein (1999) Dev Biol 212: 165-81), the chicken teneurins-1, -2 and -4 (Minet et al. (1999) J Cell Sci 112: 2019-32); (Rubin et al. (1999) Dev Biol 216:195-209); (Tucker et al. (2000) Mech Dev98, 187-91); (Tucker et al. (2001) Dev Dyn 220: 27-39) and the zebrafish Ten-m3 and Ten-m4 (Mieda et al. (1999) Mech Dev 87, 223-7) - were found to be prominently expressed in specific regions of the central nervous system.

Except for transcript localization by in situ hybridization in the nervous system and the possible involvement of teneurin-2 in the organization of the actin cytoskeleton in neuroblastoma cells, very little is known about the distribution and function of teneurins. Teneurins are known to be transmembrane proteins and are postulated to be involved in signal transduction. It has been suggested that teneurins could function as receptor proteins transmitting signals to the cell interior upon homo- or heterophilic binding of a ligand or as a membrane-bound ligand. Teneurin-2 is thought to be cleaved at the cell membrane either releasing a large part of the extracellular domain from the cell membrane, which could act as a soluble ligand or cleavage could occur after ligand binding and result in signal transduction (Rubin et al., Developmental Biology, 216,195-209, 1999).

One potential scenario by which transmembrane proteins can fulfil their role as signalling molecules is by a mechanism recently described as regulated intramembrane proteolysis (RIP; reviewed in Brown et al. (2000) Cell 100, 391-98). RIP involves at least two cleavage steps in and at the membrane resulting in a soluble cytoplasmic part, which Is translocated to the nucleus where it participates in transcription (Ebinu and Yankner (2002) Neuron 34(4):499-502. RIP is known to control diverse cellular and developmental processes. It is well known, however, that transmembrane proteins can initiate signal transduction by alternative mechanisms, such as ligand binding, receptor binding, and signalling through kinase/phosphatase cascades, etc. Indeed, Drosophila ten-m was postulated to modulate the activity of the Drosophila pair-rule gene, Odd-paired (opa) protein via a signal transduction cascade (Baumgartner et al., 1994, EMBO J. 13: 3728-3740), although a similarity between the intracellular cleavages of Odz have been likened to those of notch (Dgany and Wides, Biochem J. (2992) 363:633-643). Netherless, no evidence exists that any portion of Odz translocates to the nucleus. Furthermore, the extracellular domain of mouse Ten m1 was found to exhibit homophilic binding and thereby inittate a signal transduction pathway (Oohashi et al., 1999).

Zic genes (Zic1-4) encode zinc finger proteins homologous to opa, and are expressed in both the developing and mature CNS. In vertebrate neural development, they are generally expressed in the dorsal neural tube. A similar zinc finger gene, Opr, has also been described in mice (Furushima et al. (2000) Mech. of Development 98, 161-64). Zic1 controls the expansion of neuronal precursors by inhibiting the progression of neuronal differentiation. Notch-mediated inhibition of neuronal differentiation is likely to act downstream of Zic genes since Notch1 is upregulated in Zic1-overexpressing spinal cords in both the mouse and the chick (Aruga et al. (2002) Dev Biol. 244(2):329-41). Reduced expression of Zic2 in mice results in spina bifida and holoprosencephaly. The disruption of Zic1, a strong homolog of Zic2 that has an overlapping expression pattern, results in cerebellar malformation. It has been shown that Zic2 and Zic1 cooperatively control cerebellar development by regulating neuronal differentiation (Aruga et al. (2002) J Neurosci 22(1):218-25).

The ponsin (also known as SH3P12, CAP or FLAF2) gene encodes a protein belonging to the Ponsin/ArgBP2/venexin family. All members of this family contain three SH3 (src homology 3 region) domains. It is through these SH3 domains that Ponsin protein interacts with Vinculin, an F-actin binding protein, at cell-cell and cell-matrix adherens junctions or with Afadin at Zonula adherens (Mandai et al. (1999) J. Cell Biol. 144: 1001 -1017). Ponsin also directly interacts with the non-receptor focal adhesion tyrosine kinase p125 FAK (Ribon et al. (1 998a) J. Biol. Chem. 273: 4073-4080). Several splice variants of Ponsin mRNA exist, which are specifically up-regulated by p53 expression in EB-1 cells and by adriamycin treatment of TK6 cells (Kostic et al. (2000) Oncogene 19(35):3978-87). Loss of wild type p53 function in colon tumours coincide with migration of the tumour mass across the basement membrane.

PML (a protein named for its prevalence in promyelocytic leukemia) is a nuclear protein that controls apoptosis, cell proliferation and senescence and it is believed to be a zinc finger transcription factor. The PML gene, involved in the chromosomal translocation of acute promyelocytic leukemia (APL), encodes a protein which Is located In nuclear substructures called PML-bodies and which plays a role in tumour suppression (reviewed in Seeler et al. (1999) Curr Opin Genet Dev. 9(3):362-7; or Salomoni and Pandolfi (2002) Cell 108, 165-70).

A need exists to find signalling pathways and genes that are involved in the development of neuropathological, neurodevelopmental, and neurodegenerative diseases or conditions as well as in other therapeutic areas, such as cancer, to more accurately and effectively diagnose and treat these diseases. A more complete delineation of the teneurin signalling pathway and identification of the pathway's components provided by the present invention meets this need.

### SUMMARY OF THE INVENTION

In accordance with a first aspect of the invention a method for detecting teneurin signalling is provided, which method comprises providing (i) a teneurin or a fragment thereof, which fragment comprises at least a portion of the N-terminal domain of teneurin and at least a portion of the C-terminal domain of teneurin, and (ii) a cellular component that cleaves the teneurin; determining the presence and/or amount of a cleaved teneurin product associated with the signalling; and correlating the presence and/or amount of the cleaved teneurin product with teneurin signalling. The cleaved teneurin product comprises the cytoplasmic domain of teneurin or a fragment thereof and may be present without limitation in tumour cells containing PML nuclear bodies or in neurons. In one embodiment teneurin is teneurin-1, in another embodiment it is teneurin-2, teneurin-3, or teneurin-4.

The method of the invention for detecting teneurin signalling further comprises labelling teneurin with a detectable tag or label. In one aspect, the labelled teneurin is recombinant. In another embodiment of the present invention, the cleaved teneurin product comprises a detectable tag or label. Said tag or label may be detected photometrically and may be without limitation GFP (green fluorescence protein) or YFP (yellow fluorescence protein).

In one aspect, the determination of the presence and/or amount of a cleaved teneurin product associated with the signalling is qualitative. In a further aspect, the determination is quantitative (i.e., the amount of cleaved teneurin is detected not merely its presence).

In a further embodiment, a method is provided for detecting teneurin signalling further comprising expressing tagged teneurin from nucleic acid encoding therefor introduced into a cell or a progenitor thereof, wherein said tag may comprise a DNA binding domain which binds to nucleic acid comprising regulatory sequences operably linked to a reporter gene. Said DNA binding domain may comprise without limitation GAL 4 DNA binding domain. In another embodiment, said tag further comprises a NFκB domain. The present invention further comprises nucleic acid encoding for tagged teneurin which is stably expressed in a cell, e.g. a tumour cell or a neuron.

In a further aspect, the present invention provides a method for detecting teneurin signalling wherein the cleaved teneurin product regulates expression or activity of a cellular target which itself is a modulator of cell proliferation or neuronal differentiation, e.g. PML, zic, ponsin, p53 and myc, or wherein the cleaved teneurin product targets to the nucleus. Said cellular target of the cleaved teneurin product may be detected by expression array analysis.

Also provided is a method for detecting teneurin signalling wherein the presence and/or amount of the detectable cleaved teneurin product is correlated to a particular disease. This method can be performed for determining whether a cleaved teneurin product is involved in cell proliferation or neuronal differentiation.

In one embodiment, an in vitro method of diagnosis of a neuropathology or cell pathology which affects teneurin-mediated signalling is provided which comprises performing the hereinabove described methods of the invention on a cell which has been extracted from an animal which it is desired to diagnose.

In another embodiment, the use of a detectable cleaved teneurin product associated with teneurin signalling in an in vivo method of diagnosis of a neuropathology or cell pathology which affects teneurin signalling is provided, which method comprises performing a method of the invention as hereinabove described.

The invention further provides a method for assessing the ability of an agent to modulate teneurin signalling, comprising the steps of: (a) contacting teneurin with at least one agent in the presence of a cellular component; (b) detecting cleavage of said teneurin in the presence or absence of said agents by use of a method of the invention; and (c) correlating the value obtained in step (b) with a value obtained in the absence of said agent, and correlating a difference between values as an indication of the presence of an agent effective in modulating teneurin signalling.

In another aspect, a method for assessing the ability of an agent to modulate teneurin signalling is provided further comprising: (a) determining the presence and/or amount of cleaved teneurin product in a cell; (b) exposing the cell expressing teneurin to the agent; (c) measuring the presence and/or amount of cleaved teneurin product in the presence of the agent; and (d) comparing the determinations made in (a) and (c). Also provided is a method wherein step (b) is performed by perfusing the cell with the agent.

In another aspect, a method for assessing the ability of an agent to modulate teneurin-mediated signalling is provided, comprising the steps of: (a) measuring expression or activity of genes regulated by teneurin in cells; (b) exposing said cells to the agent; (c) measuring expression or activity of genes regulated by teneurin in said cells in the presence of the agent; and (d) correlating a modulated value in step (a) compared to step (c) with the ability of the agent to regulate teneurin signalling.

Also provided is a method of screening for compounds for the potential to modulate cell proliferation or for compounds for the treatment of neurodegenerative disease or disorder or for the treatment of cancer, which method comprises assessing the ability of said compounds to modulate teneurin signalling by use of a method of the invention. In another embodiment, a method of screening is provided further comprising the subsequent step of isolation and/or manufacture and/or use in a method of treatment, of an agent which tests positive.

In a further aspect, a composition detected by a method of screening of the invention is provided for the treatment or prophylactic treatment of tumourigenesis or cancer or for the treatment or prophylactic treatment of neuropathology. The composition detected by a method of screening can be used for the manufacture of a medicament for the treatment or prophylactic treatment of tumourigenesis or cancer or for the manufacture of a medicament for the treatment or prophylactic treatment of neuropathology.

Also provided Is the use of a cleaved teneurin product for the manufacture of a medicament for the treatment or prophylactic treatment of tumourigenesis or cancer or neuropathology.

Further provided is a composition comprising a cleaved teneurin product and a cellular target of the cleaved teneurin product, such as PML, Zic, p53, myc or ponsin.

### DETAILED DESCRIPTION OF THE INVENTION

Intracellular pathways are known to be regulated by a cascade of components that undergo modulation in a temporally and spatially characteristic manner. Several disease states can be attributed to altered activity of individual signalling components (e.g., protein kinases, protein phosphatases and transcription factors). These components therefore render themselves as attractive targets for therapeutic intervention, or as targets for agents that seek to obtain a biological effect by modulating a signalling pathway. Typically, modulation of a signalling pathway will alter the response of a cell to a particular stimulus in a variety of ways. Although much work has been carried out in attempting to delineate the involvement of proteins in signalling pathways, prior to the present invention no hypothesis was put forward linking teneurin to components of a signalling pathway.

The present inventors have demonstrated for the first time that teneurin is involved in intracellular signalling by its cytoplasmic domain interacting with cellular targets that influence gene transcription and are involved in cerebellar development, neuronal differentiation, apoptosis, cell proliferation and senescence. Modulation of teneurin signalling to these targets may therefore have application in neuropsychiatric, neurodevelopmental, and neurodegenerative diseases and other CNS diseases or conditions, as well as for the understanding of neuronal differentiation, cell proliferation and oncogenesis.

The present inventors have found that teneurin is involved in intracellular signalling and exhibits transcriptional activity through its cytoplasmic domain. The cytoplasmic domain of teneurin-2 can be released from the cell membrane and translocates to the cell nucleus where it is able to influence the transcription activity of various target genes including zic-1, a vertebrate homologue of the Drosophila opa. Teneurin signalling may be of importance in many neurodevelopmental and neurodegenerative diseases and disorders, such as schizophrenia, Alzheimer's, Parkinsons disease, amyloid angiopathies, and other developmental disorders such as spina bifida and mental retardation. In addition teneurin Is thought to be important in adhesion, signalling, regulating cell survival, proliferation and differentiation and teneurin signalling may have applications in cell proliferation, such as in cancer.

Zic1, is a zinc finger transcription factor homologous to the Drosophila pair-rule gene opa, a possible downstream target of one of the Drosophila teneurins.

The present inventors have investigated the role of teneurin in intracellular signalling and found intracellular teneurin targets. Using live Imaging of cells expressing labelled or tagged proteins, the inventors showed that transfection of cells with a construct expressing the cytoplasmic domain of teneurin-2 resulted in the translocation of the cytoplasmic domain to the nucleus of the cells. Its expression was confined to discrete spots within the nucleus in contrast to the staining pattern of teneurin comprising the transmembrane domain of teneurin-2, which showed accumulation on the cell surface (Example 1). The nuclear localisation coincided with a very similar punctuate pattern obtained by staining for PML (promyelocytic leukemia), a protein which localizes to the PML-nuclear bodies and which controls apoptosis, cell proliferation, and scenescence. An identical staining pattern was detected after co-transfection of the cytoplasmic domain of teneurin-2 with PML, suggesting an involvement of the cytoplasmic domain of teneurin-2 in transcriptional regulation.

Furthermore, the present inventors have demonstrated that co-transfection of the cytoplasmic domain of teneurin-2 with Zic1 reduced the transcriptional activity of Zic which suggests a role of teneurin in the control of cerebellar development by regulating neuronal differentiation and other neurological conditions.

In a yeast-two-hybrid screen the cytoplasmic domain of teneurin-1 was found to bind to ponsin, a p53 responsive gene also involved in apoptosis and scenescence. The present inventors confirmed this binding by co-transfection and co-immunoprecipitation of the two proteins.

Microarray analysis was performed using Affymetrix HG-U133A GeneChips™ to identify genes that are regulated by teneurin signalling. Among the genes up- and down-regulated by teneurin-2, many were shown to play a role in the regulation of cell growth, differentiation and apoptosis. A group of genes down-regulated by teneurin-2 correlated with genes known to be glutathionylated and involved in the regulation of the redox status of cells. Many of the teneurin-2 up-regulated genes are targets of p53, whereas many of the teneurin-2 down-regulated genes are targets of myc.

Together these data indicate that teneurin plays a role In intracellular signalling pathways which has not been known from previous studies. In general terms, the present invention relates to a cleaved teneurin product as a specific marker for teneurin-mediated signalling.

Accordingly, a method for detecting teneurin signalling is provided, which method comprises (a) determining the presence and/or amount of a cleaved teneurin product associated with the signalling; and (b) correlating the presence and/or amount of the cleaved teneurin product with teneurin signalling. Optionally, the method further comprises providing a teneurin or a fragment thereof comprising at least a portion of the N-terminal domain of teneurin and at least a portion of the C-terminal domain of teneurin, and a cellular component that cleaves teneurin.

By ‘signalling’ is meant a process regulated by a cascade of components that undergoes modulation in a temporally and spatially characteristic manner (Gomperts et al., (2002), Signal transduction, San Diego, Academic Press, 424 p.; or Krauss (2001), Biochemistry of Signal Transduction and Regulation, Wiley-VCH, 2nd edition, 528 p.). Activity of individual intracellular signalling components (e.g., protein kinases, protein phosphatases, transcription factors) can be altered in several disease states. Teneurin signalling can be detected by analysing the modulation (up- or down-regulation) of downstream targets of teneurin, which play a role in the regulation of cell growth, differentiation and apoptosis.

The teneurin protein provided for the method of the present invention may be mammalian teneurin, preferably mouse or rat teneurin, more preferably human teneurin. It may be chosen from teneurin-1, teneurin-2, teneurin-3 or teneurin-4 (described in Minet and Chiquet-Ehrismann (2000) Gene 257: 87-97).

By a ‘fragment’ of teneurin is meant a fragment comprising a protease cleavage site and at least a portion of the exodomain of teneurin, a transmembrane domain, and at least a portion of the cytoplasmic domain of teneurin. The portion will typically be at least one, preferably at least ten or more amino acid residues in length.

A ‘cellular component’ in the meaning of the present invention has a protease activity and Is capable of cleaving teneurin to provide a fragment capable of signalling and affecting teneurin targets. The activity may regulate intramembrane proteolysis (RIP), where at least two cleavage steps by proteases lead to the separation of the intracellular part from the membrane. The first cleavage and splitting off of the truncated extracellular or intraluminal parts are prerequisite for the second cleavage. The latter takes place within the transmembrane domain. The resulting soluble cytoplasmic part is subsequently translocated to the nucleus where it participates in transcription (reviewed in Brown et al. (2000) Cell 100, 391-98). The cellular component of the present invention may thus work in conjunction with another protease activity, but comprises at least one protease capable of cleaving teneurin in or at the transmembrane domain resulting in a soluble cleaved teneurin product associated with teneurin signalling. The protease may be selected from the group including, without limitation, site 1 proteases, site 2 proteases, or alpha-, beta- or gamma-secretase. These proteases have been shown to be involved in the regulated intramembrane proteolysis (RIP) mediated nuclear signalling (Ebinu and Yankner (2002) Neuron 34(4):499-502).

The cleaved teneurin product of the invention comprises the cytoplasmic domain of teneurin or a fragment thereof and may be present, without limitation, in tumour cells containing PML nuclear bodies or in neurons. The cytoplasmic domain of teneurin may be a polypeptide having the amino acid sequence shown in SEQ ID NO: 2, SEQ ID NO: 4, SEQ ID NO: 6, or SEQ ID NO: 8, or a variant thereof such as a variant comprising an amino acid deletion, addition (e.g. fusion proteins) or substitution relative to the above mentioned amino acid sequences. The various nucleic acids that can encode these polypeptides therefore may differ because of the degeneracy of the genetic code, in that most amino acids are encoded by more than one triplet codon. The identity of such codons is well known in this art, and this information can be used for the construction of the nucleic acids within the scope of the invention. Variants differ from wild-type protein in having one or more amino acid substitutions that enhance, add, or diminish a biological activity of the wild-type protein.

By a ‘fragment of the cytoplasmic domain of teneurin’ is meant a fragment of a polypeptide having a number of amino acid residues in the range of 5-400, more preferably 10-300, most preferably 20-200.

Exemplary functional equivalents or derivatives of the amino acid sequences shown in SEQ ID NO: 2, SEQ ID NO: 4, SEQ ID NO: 6, or SEQ ID NO: 8 include molecules wherein the polypeptide of the invention is covalently modified by substitution, chemical, enzymatic, or other appropriate means with a moiety other than a naturally occurring amino acid. Derivatives which retain common structural features can be fragments of the protein of the Invention, in particular fragments maintaining teneurin signalling acitivity.

Useful fragments may exhibit an epitope recognized by polyclonal or monoclonal antibodies raised against the polypeptide having the amino acid sequence shown in SEQ ID NO: 2, SEQ ID NO: 4, SEQ ID NO: 6, or SEQ ID NO: 8 for example. Particularly preferred fragments are those exhibiting a sequence capable of inducing or inhibiting teneurin signalling.

The determination step in the method may be preceded by exposing the cell or the cell free system to ponasterone or to another putative or known activating stimulus, including without limitation pharmacological agents, to induce expression of teneurin.

In some embodiments, the method for detecting teneurin signalling further comprises labelling teneurin or the cleaved teneurin product with a detectable tag or label. In one aspect, the labelled teneurin is recombinant, such as in the form of a fusion with another protein, for example, tags for the targeted delivery or detection of the polypeptide (including fragments thereof). It is desirable that the tag or label is easily detectable. In principle it may be inherently detectable (e.g. a protein identifiable by a consonant antibody). Immunological methods of detection include, Western blot, immunohistochemical staining of cells or tissue sections, and assays of cell culture, to quantitate directly the expression of gene product. Antibodies useful for immunohistochemical staining and/or assay of sample fluids may be either monoclonal or polyclonal, and may be prepared by conventional methods well known in the art. Conveniently, the antibodies may be prepared against a native sequence of the tag or label.

In preferred embodiments the ‘label’ in the meaning of the present invention will include a specific detectable label for increased ease of scoring and/or sensitivity. Most preferably the activity of the labelled protein, or the protein itself, can be estimated photometrically (especially by fluorimetry or luminometry). This may be directly e.g. using for instance green fluorescent protein (GFP), yellow fluorescent protein (YFP), insect luciferase or photobacterial luciferase. Alternatively, a radioactive or phosphorescent label may be used.

In another preferred embodiment, a ‘tag’ may be used wherein the detection may be indirect e.g. whereby the signal gene causes a change which is detected by a colour indicator e.g. on staining. Other suitable signal proteins (which have a readily detectable activity) are known in the art e.g. β-galactosidase, which can generate a coloured product from its substrate. The signal may utilise co-factors. The tag may alternatively comprise a DNA binding domain which binds to nucleic acid comprising regulatory sequences operably linked to a reporter gene. The DNA binding domain may be, for example and without limitation, the GAL 4 DNA binding domain. In another embodiment, the tag may further comprise an NFκB domain.

The method thus encompasses the detection of teneurin signalling by means of a cleaved teneurin product which is associated with the signalling and a detectable tag or label in that the presence and\or amount of the cleaved teneurin product is increased when teneurin signalling is activated. In general the tag or label under the conditions used in the method, will not be present (or detectably present) prior to contacting the cell or the cell free system with another cellular component, a protease. The determination of the presence and\or amount of the detectable tag or label attached to the cleaved teneurin product may be either qualitative or quantitative, and the correlation may be based on comparison with the tag or label in unactivated systems (either directly, or based on historical or contemporaneous comparison). For instance the amount of detectable cleaved teneurin product may be scored in each case and the scores compared.

In order to produce suitable test systems including detectable cleaved teneurin product, it will generally be preferred to use nucleic acid encoding teneurin, which is either stably or transiently expressed. A nucleic acid encoding teneurin or its labelled or tagged variants may therefore be introduced into a cell or a progenitor thereof to obtain expression of the protein. Preferably, the teneurin is stably expressed in a cell. Alternatively, cells expressing teneurin can be used, such as tumour cells, neurons or a progenitors thereof. In a preferred embodiment, cultured tumour cells are used. Alternatively, the teneurin may be employed by in a cell free system and allowing detection with an antibody or using protein-protein interactions, for example.

Nucleic acids of, or for use in, the present invention (e.g. encoding labelled teneurin) may be provided isolated from their natural environment, in substantially pure or homogeneous form, or free or substantially free of other nucleic acids of the species of origin. Where used herein, the term “isolated” encompasses all of these possibilities. Nucleic acid according to the present invention may be in the form of, or derived from, cDNA, RNA, genomic DNA and modified nucleic acids or nucleic acid analogs. Thus the invention also relates, in a further aspect, to use of a nucleic acid molecule which comprises a nucleotide sequence encoding teneurin described above linked to a detectable label, in the various methods of the Invention.

Nucleic acid sequences which encode a tagged polypeptide or peptide linked to a label in accordance with the present invention can be readily prepared by the skilled person using the information and references contained herein and techniques known in the art (for example, see Sambrook, Fritsch and Maniatis, “Molecular Cloning, A Laboratory Manual”, Cold Spring Harbor Laboratory Press, 1989, and Ausubel et al., Short Protocols in Molecular Biology, John Wiley and Sons, 1992). These techniques include (i) the use of the polymerase chain reaction (PCR) to amplify samples of the relevant nucleic acid, e.g. from genomic sources, (ii) chemical synthesis, or (iii) preparation of cDNA sequences.

Thus in cell-based assays of the present invention, the labelled protein of interest can be introduced by causing or allowing the expression in a cell of an expression construct or vector. The construct may include any other regulatory sequences or structural elements as would commonly be included in such a system, and as is described below. As well as the signal sequence, the vector components will usually include, but are not limited to, one or more of an origin of replication, one or more marker genes, an enhancer element, a promoter, and a transcription termination sequence. Construction of suitable vectors containing one or more of these components employs standard ligation techniques which are known to the skilled artisan.

Nucleic acid sequences which enable a vector to replicate in one or more selected host cells are well known for a variety of bacteria, yeast, and viruses. For Example, various viral origins (SV40, polyoma, adenovirus, VSV or BPV) are useful for cloning vectors in mammalian cells. Particularly preferred is an expression vector comprising a nucleic acid as described herein. The vector may, for example, be in the form of a plasmid, cosmid, viral particle, phage, or any other suitable vector or construct which can be taken up by a cell and used to express the detectable marker.

Expression vectors usually contain a promoter operably linked to the protein-encoding nucleic acid sequence of interest, so as to direct mRNA synthesis. Promoters recognized by a variety of potential host cells are well known. “operably linked” means joined as part of the same nucleic acid molecule, suitably positioned and oriented for transcription to be initiated from the promoter. DNA operably linked to a promoter is “under transcriptional control” of the promoter. Transcription from vectors In mammalian host cells is controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g. the actin promoter or an immunoglobulin promoter, and from heat-shock promoters, provided such promoters are compatible with the host cell systems.

Expression vectors of the Invention may also contain one or more selection genes. Typical selection genes encode proteins that (a) confer resistance to antibiotics or other toxins e.g. ampicillin, neomycin, methotrexate, or tetracycline, (b) complement auxotrophic deficiencies, or (c) supply critical nutrients not available from complex media, e.g., the gene encoding D-alanine racemase for Bacilli.

The methos of the invention may therefore further include introducing the nucleic acid into a host cell. The introduction, which may (particularly for in vitro introduction) be generally referred to without limitation as “transformation”, may employ any available technique. For eukaryotic cells, suitable techniques may include calcium phosphate transfection, DEAE-Dextran, electroporation, liposome-mediated transfection and transduction using retrovirus or other virus, e.g. vaccinia or, for insect cells, baculovirus. For example, the calcium phosphate precipitation method of Graham and van der Eb, Virology 52:456-457 (1978) can be employed. General aspects of mammalian cell host system transformations have been described in U.S. Pat. No. 4,399,216. For various techniques for transforming mammalian cells, see Keown et al., Methods in Enzymology, 185:527 537 (1990) and Mansour et al., Nature 336:348-352 (1988).

Host cells transfected or transformed with expression or cloning vectors described herein may be cultured in conventional nutrient media. The culture conditions, such as media, temperature, pH and the like, can be selected by the skilled artisan without undue experimentation. In general, principles, protocols, and practical techniques for maximizing the productivity of cell cultures can be found in “Mammalian Cell Biotechnology: a Practical Approach”, M. Butler, ed. JRL Press, (1991) and Sambrook et al, supra.

There is no requirement that labelled teneurin of the present invention has to include the full-length sequence of the protein as it occurs in nature. Variants may be used (e.g. which are derived from teneurin) which retain its activity. The term “derived” includes variants produced by modification of the authentic native sequence e.g. by introducing changes into the full-length or truncated sequence, for example substitutions, Insertions, and/or deletions. This may be achieved by any appropriate technique, Including restriction of the coding sequence with an endonuclease followed by the Insertion of a selected base sequence (using linkers if required) and ligation. Also possible is PCR-mediated mutagenesis using mutant primers. It may, for instance, be preferable to add in or remove restriction sites in order to facilitate further cloning.

Modified sequences according to the present invention may have a sequence at least 60% identical to the sequence of teneurin. Typically there would be 80% or more, 90% or more 95% or more or 98% or more identity between the modified sequence and the authentic sequence. There may be up to five, for example up to ten or up to twenty or more nucleotide or amino acid deletions, insertions and/or substitutions made to the full-length or part length sequence provided functionality is not totally lost. Similarity or identity may be as defined and determined by the TBLASTN program, of Altschul et al. (1990) J. Mol. Biol. 215: 403-10, or BestFit, which is part of the Wisconsin Package, Version 8, Sep. 1994, (Genetics Computer Group, 575 Science Drive, Madison, Wis., USA, Wisconsin 5371 1). Preferably sequence comparisons are made using FASTA and FASTP (see Pearson & Lipman, 1988. Methods in Enzymology 183:63-98). Parameters are preferably set, using the default matrix, as follows: Gapopen (penalty for the first residue in a gap): -16 for DNA; Gapext (penalty for additional residues in a gap): -4 for DNA KTUP word length: 6 for DNA. Alternatively, homology in the context of nucleic acids can be judged by probing under appropriate stringency conditions. One common formula for calculating the stringency conditions required to achieve hybridization between nucleic acid molecules of a specified sequence homology is (Sambrook et al., 1989): Tₘ=81.5° C.+16.6 Log [Na+]+0.41 (% G+C)−0.63 (% formamide)−600/#bp in duplex.

The present inventors have demonstrated for the first time that the cytoplasmic domain of teneurin, the cleaved teneurin product of the invention, contains transcriptional activity or acts as a transcriptional modulator which are characteristics of proteins regulated by RIP (regulated intramembrane proteolysis). It could be found that the cytoplasmic domain of teneurin translocates to the nucleus and is capable of interacting with a cellular target. These targets are found to include without limitation proteins belonging to the ponsin/ArgBP2/venexin family (including without limitation ponsin, SH3P12, CAP, SORB1, ArgBP2, venexin, nArgBP2, FLAF2), preferably ponsin (see Example 3), nuclear targets such as the nuclear protein PML (promyelocytic leukemia, see Example 1), or transcription factors such as zinc finger proteins which include without limitation Zic1-4, or Opr, preferably Zic1 (see Example 2), as well as other targets that are involved in neuronal differentiation or cell proliferation detected by expression array analysis (Example 5, Table 1 and 2) which might include without limitation neuroserpin, protein kinase C (PKC) or any other hitherto unknown target of teneurin.

In a further aspect, the present Invention provides compositions capable of enhancing or inhibiting expression or activity of the cellular targets that regulate cell proliferation or neuronal differentiation. Therefore, depending on the function of its intracellular target, the cleaved teneurin product of the present invention is indirectly capable of enhancing or inhibiting transcriptional activation.

In a further aspect, the present invention provides a method for detecting teneurin signalling wherein the cleaved teneurin product regulates expression or activity of a cellular target which itself is a modulator of cell proliferation or neuronal differentiation, which may include, without limitation, PML, Zic, ponsin, p53-regulated genes, myc-regulated genes, or wherein the cleaved teneurin product targets to the nucleus. The cellular target of the cleaved teneurin product may be detected by expression array analysis.

Also provided is a method for detecting teneurin signalling wherein the presence and/or amount of the detectable cleaved teneurin product is correlated to a particular disease. This method can be performed for determining whether a cleaved teneurin product is involved in cell proliferation or neuronal differentiation.

A ‘disease’ correlated to the cleaved teneurin product of the invention may mean any disease wherein cell proliferation or neuronal differentiation is affected, any cell pathology or neuropathology including, without limitation, neurodevelopmental and neurodegenerative diseases and disorders such as schizophrenia, multiple sclerosis, Alzheimer's, Parkinsons disease, amyloid anglopathies, and other developmental disorders such as spina bifida or even mental retardation, as well as cancer. Targets of the cleaved teneurin product of the invention have been shown to be involved in tumourigenesis (e.g. ponsin which is up-regulated by p53 expression, described in Kostic et al. (2000) Oncogene 19(35):3978-87), In neuronal differentiation and cerebellar development (reduced expression of zic results in spina bifida and holoprosencephaly, described in Aruga et al. (2002) Dev Biol. 244(2):329-41), or in cell proliferation or apoptosis (PML, described in Salomoni and Pandolfi (2002) Cell 108 165-70).

In one embodiment, an in vitro method of diagnosis of a neuropathology or cell pathology which affects teneurin-mediated signalling is provided which comprises performing the hereinabove described methods of the invention on a cell which has been extracted from an animal which it Is desired to diagnose.

The cleaved teneurin product disclosed herein may be used as a diagnostic. The expression of the cleaved teneurin product or the expression or activity of target genes of the cleaved teneurin product may be correlated to a particular disease state (which may include, without limitation, schizophrenia, Alzheimer's, Parkinsons disease, amyloid angiopathies, spina bifida, multiple sclerosis, mental retardation, or cancer) and based on the diagnosis particular therapies could be chosen. Thus the invention provides methods of diagnosis and use of the materials disclosed herein in such methods, particularly in respect of neuropathologies or cancer.

The invention further provides the use of a detectable cleaved teneurin product associated with teneurin signalling in an in vivo method of diagnosis of a neuropathology or cell pathology which affects teneurin signalling is provided, which method comprises performing a method of the invention as hereinabove described.

For in vivo methods, host cells according to the present invention (i.e. including the detectable teneurin) may be comprised in a transgenic animal, and the present invention further provides for a transgenic animal, comprising cells which express a labelled teneurin, such as a fusion protein of teneurin with a fluorescent protein, and also uses thereof. The transgenic organisms of the invention all include within a plurality of their cells a cloned recombinant or synthetic DNA sequence which encodes the labelled teneurin.

Since it is possible to produce transgenic organisms of the invention utilizing a variety of labels and tags, a general description will be given of the production of transgenic organisms by referring generally to exogenous genetic material. This general description can be adapted by those skilled in the art in order to incorporate the above-described specific DNA sequences into organisms and obtain expression of those sequences utilizing the methods and materials described below. For more details regarding the production of transgenic organisms, and specifically transgenic mice, refer to U.S. Pat. No. 4,873,191, issued Oct. 10, 1989 (incorporated herein by reference to disclose methods producing transgenic mice), and to the numerous scientific publications referred to and cited therein.

The exogenous genetic material may be placed in either the male or female pronucleus of the zygote. More preferably, it is placed in the male pronucleus as soon as possible after the sperm enters the egg. In other words, right after the formation of the male pronucleus when the pronuclei are clearly defined and are well separated, each being located near the zygote membrane. The male pronucleus of a fertilized mouse egg is the preferred site for addition of the exogenous genetic material of the present invention.

It is most preferred that the exogenous genetic material be added to the male DNA complement of the zygote prior to its being processed by the ovum nucleus or the zygote female pronucleus. It is thought that the ovum nucleus or female pronucleus release molecules which affect the male DNA complement, perhaps by replacing the protamines of the male DNA with histones, thereby facilitating the combination of the female and male DNA complements to form the diploid zygote.

Thus, it is preferred that the exogenous genetic material be added to the male complement of DNA or any other complement of DNA prior to its being affected by the female pronucleus. For example, the exogenous genetic material is added to the early male pronucleus, as soon as possible after the formation of the male pronucleus, which is when the male and female pronuclei are well separated and both are located close to the cell membrane. Alternatively, the exogenous genetic material could be added to the nucleus of the sperm after it has been induced to undergo decondensation. Sperm containing the exogenous genetic material could then be added to the ovum or the decondensed sperm could be added to the ovum with the exogenous genetic material being added as soon as possible thereafter.

For the purposes of this invention a zygote is essentially the formation of a diploid cell which is capable of developing into a complete organism. Generally, the zygote will be comprised of an egg containing a nucleus formed, either naturally or artificially, by the fusion of two haploid nuclei from a gamete or gametes. Thus, the gamete nuclei must be ones which are naturally compatible, I.e., ones which result in a viable zygote capable of undergoing differentiation and developing into a functioning organism. Generally, a euploid zygote is preferred. If an aneuploid zygote is obtained, then the number of chromosomes should not vary by more than one with respect to the euploid number of the organism from which either gamete originated.

In addition to similar biological considerations, physical ones also govern the amount of exogenous genetic material which can be added to the nucleus of the zygote or to the genetic material which forms a part of the zygote nucleus. If no genetic material is removed, then the amount of exogenous genetic material which can be added is limited by the amount which will be absorbed without being physically disruptive. Generally, the volume of exogenous genetic material inserted will not exceed about 10 picoliters. The physical effects of addition must not be so great as to physically destroy the viability of the zygote. The biological limit of the number and variety of DNA sequences will vary depending upon the particular zygote and functions of the exogenous genetic material and will be readily apparent to one skilled in the art, because the genetic material, including the exogenous genetic material, of the resulting zygote must be biologically capable of initiating and maintaining the differentiation and development of the zygote into a functional organism.

The number of copies of the DNA sequences which are added to the zygote is dependent upon the total amount of exogenous genetic material added and will be the amount which enables the genetic transformation to occur. Theoretically only one copy is required; however, generally, numerous copies are utilized, for example, 1,000-20,000 copies of a gene, in order to insure that one copy is functional. As regards the present invention, there is generally an advantage to having more than one functioning copy of each of the inserted exogenous DNA sequences to enhance the phenotypic expression of the exogenous DNA sequences.

Any technique which allows for the addition of the exogenous genetic material into nucleic genetic material can be utilized so long as it is not destructive to the cell, nuclear membrane or other existing cellular or genetic structures. The exogenous genetic material is preferentially inserted into the nucleic genetic material by microinjection. Microinjection of cells and cellular structures is known and is used in the art.

Thus the present invention provides methods in which cloned recombinant DNA sequences encoding appropriate membrane targeting sequences may be injected Into fertilized mammalian eggs (preferably mouse eggs). The injected eggs are implanted in pseudo pregnant females and are grown to term to provide transgenic mice whose cells express proteins related to the pathology of the relevant disease. The injected sequences are constructed having promoter sequences connected so as to express the desired protein in specific tissues of the transgenic mammal (most notably in nerve tissue). Examples may include the prion specific promoter of Lewis et al (2000) Nature Genetics 25, 402-405, or the neurospecific enolase promoter.

Non-human animals of the invention may be homozygous or heterozygous for the fusion polypeptide. Mammalian animals include non-human primates, rodents, rabbits, sheep, cattle, goats, pigs. Rodents include mice, rats, and guinea pigs.

Transgenic non-human mammals of the invention may, inter alia, be used for experimental purposes in studying neuronal development or cell proliferation and the development of therapies designed to alleviate the symptoms of disease in which improper teneurin signalling is implicated. By “experimental” it is meant permissible for use in animal experimentation or testing purposes under prevailing legislation applicable to the research facility where such experimentation occurs.

An important aspect of the present invention is the use of the detectable teneurin or of the detectable cleaved teneurin product disclosed herein to screen for modulators affecting teneurin signalling, for example modulators (e.g. agonists or antagonists) affecting transcription of genes regulated by teneurin and therefore affecting cell proliferation, apoptosis, neuronal differentiation, epilepsy, neurodegeneration, multiple sclerosis, schizophrenia, Alzheimer's, Parkinsons disease, amyloid angiopathies, and other developmental disorders such as spina bifida or even mental retardation. This can be achieved in the light of the disclosure herein by looking for compounds that would enhance (for agonists) or supress (for antagonists) expression of the cleaved teneurin product (or cause activation or redistribution of other proteins that are regulated by teneurin), or that would enhance or supress protease activity of proteases that cleave teneurin. Thus, an indicator for the cleaved teneurin product is useful for screening for compounds that improve cancerous conditions or induce neuronal regeneration.

Accordingly, the present invention provides a method for screening compounds for the modulation of teneurin signalling comprising assaying for a modification of cleaved teneurin product, optionally labelled, and targeting to the nucleus of a cell, such a s a transcription factor.

Thus, the invention provides a method for assessing the ability of an agent to modulate teneurin signalling, comprising the steps of: (a) contacting teneurin with at least one agent in the presence of one or more cellular components (capable of cleaving teneurin); (b) detecting cleavage of said teneurin in the presence or absence of said agent by use of a method of the invention as hereinabove described; and (c) correlating the value obtained in step (b) with a value obtained in the absence of said agent, and correlating a difference between values as an indication of the presence of an agent effective in modulating teneurin signalling. An increase in cleaved teneurin product correlates with an agent that is an agonist of teneurin signalling or an agonist of a protease involved in the cleavage process of teneurin and may therefore be used as an anti-tumour agent. A decrease In cleaved teneurin product correlates with an agent that is an antagonist of teneurin signalling or an antagonist of a protease involved in the cleavage process of teneurin and may therefore be used as an agent inhibiting neuronal degeneration.

Thus the various methods described above may comprise the further steps of contacting the cleaved teneurin product, or proteases cleaving teneurin, with one or more agents which it is desired to assess for ability to modulate teneurin signalling, and comparing the signalling in the presence or absence of said agents—the relative values may be correlated with its activity as a modulator.

Therefore, a method for assessing the ability of an agent to modulate teneurin signalling is provided further comprising: (a) determining the presence and/or amount of cleaved teneurin product in a cell; (b) exposing the cell expressing teneurin to the agent; (c) measuring the presence and/or amount of cleaved teneurin product in the presence of the agent; and (d) comparing the determinations made in (a) and (c). Also provided is a method wherein step (b) is performed by perfusing the cell with the agent.

In another aspect, a method for assessing the ability of an agent to modulate teneurin-mediated signalling is provided, comprising the steps of: (a) measuring expression or activity of genes regulated by teneurin in cells; (b) exposing said cells to the agent; (c) measuring expression or activity of genes regulated by teneurin in said cells in the presence of the agent; and (d) correlating a modulated value in step (a) compared to step (c) with the ability of the agent to regulate teneurin signalling.

Also provided is a method of screening for agents for the potential to modulate cell proliferation for the treatment of cancer or diabetes or for agents for the treatment of neurodegenerative disease or disorder, which method comprises assessing the ability of said agents to modulate teneurin signalling by use of a method of the invention.

Where the method of Identifying modulators utilizes a cell-based system, it may further include the step of testing the viability of the cells expressing the labelled cleaved teneurin product e.g. by use of a lactate dehydrogenase assay kit (Sigma). This step may provide an indication of any interference by the test agent of vital cellular functions. Essentially, methods of the present invention may be employed analogously to high throughput screens such as those well known in the art—see e.g. WO 200016231 (Navicyte); WO 200014540 (Tibotec); DE 19840545 (Jerini Biotools); WO 200012755 (Higher Council for Scientific Research); WO 200012705 (Pausch M H; Wess J); WO 200011216 (Bristol-Myers Squibb); U.S. Pat. No. 6,027,873 (Genencor Intl.); DE 19835071 (Carl Zeiss; F Hoffman-La Roche); WO 200003805 (CombiChem); WO 200002899 (Biocept); WO 200002045 (Euroscreen); U.S. Pat. No. 6,007,690 (Aclara Biosciences).

Agents which are tested may be any which it is desired to assess for the relevant signalling.

The methods can serve either as primary screens, in order to identify new inhibitors/modulators, or as secondary screens in order to study known inhibitors/modulators in further detail.

‘Agents’ may be natural or synthetic chemical compounds. Relatively small chemical compounds, preferably which are capable of crossing the blood-brain barrier, may be preferred. Inhibition of the proteases involved in the separation of the intracellular part of teneurin from the membrane part can block signalling and therefore inhibit or activate transcription. Specific protease inhibitors could therefore be particularly useful as potential agents for inhibting teneurin signalling and could be used as potent tools to prevent generation of signalling products or as specific targets for cancer therapy.

The skilled person will appreciate that the amount of test substance or compound which is added in a screening assay according to this aspect of the invention will normally be determined by trial and error depending upon the type of compound used. It may be selected to be a level which could realistically be used in therapeutic context i.e. would be non-lethal to a patient. Typically, from about 0.01 to 100 nM concentrations of putative modulator compound may be used, for example from 0.1 to 10 nM.

In a further aspect, a composition detected by a method of screening of the invention is provided for the treatment or prophylactic treatment of tumourigenesis or cancer or for the treatment or prophylactic treatment of neuropathology. The composition detected by a method of screening can be used for the manufacture of a medicament for the treatment or prophylactic treatment of tumourigenesis or cancer or even diabetes, or for the manufacture of a medicament for the treatment or prophylactic treatment of neuropathology or the inhibition of neuronal degeneration.

Performance of a screening assay method according to the various aspects above may be followed by isolation and/or manufacture and/or use of a compound, substance or molecule which tests positive for ability to interfere with or modulate the neuronal differentiation, neuronal development or cell proliferation.

The compounds thus identified may be formulated into compositions for use in the diagnosis, prognosis or therapeutic treatment. Thus, the present invention also extends, in further aspects, to pharmaceutical formulations comprising one or more inhibitory or modulatory compound as obtainable by a screening method as provided herein.

A compound which has been identified as described above, may be manufactured and/or may be used in the preparation, i.e. the manufacture or formulation, of a composition such as a medicament, pharmaceutical composition or drug. These may be administered to individuals.

Not only the use of compounds which have been identified by a method of screening of the invention, but also the use of a cleaved teneurin product is provided for the manufacture of a medicament for the treatment or prophylactic treatment of tumourigenesis or cancer or neuropathology.

Whether the compound which has been identified as described above is a polypeptide, antibody, peptide, nucleic acid molecule, small molecule, mimetic or other pharmaceutically-useful compound according to the present invention, or a cleaved teneurin product that is to be given to an individual, administration is preferably in a “prophylactically effective amount” or a “therapeutically effective amount” (as the case may be, although prophylaxis may be considered therapy), this being sufficient to show benefit to the individual. The actual amount administered, and rate and time-course of administration, will depend on the nature and severity of what is being treated. Prescription of treatment, e.g. decisions on dosage etc, is within the responsibility of general practitioners and other medical doctors.

Pharmaceutical compositions according to the present invention, and for use in accordance with the present invention, may include, in addition to active ingredient, a pharmaceutically acceptable excipient, carrier, buffer, stabiliser or other materials well known to those skilled in the art. Such materials should be non-toxic and should not interfere with the efficacy of the active ingredient. The precise nature of the carrier or other material will depend on the route of administration, which may be oral, or by injection, e.g. cutaneous, subcutaneous or intravenous.

Pharmaceutical compositions for oral administration may be in tablet, capsule, powder or liquid form. A tablet may include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

For intravenous, cutaneous or subcutaneous injection, or injection at the site of affliction, the active ingredient will be in the form of a parenterally acceptable aqueous solution which is pyrogen-free and has suitable pH, Isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

Examples of techniques and protocols mentioned above can be found in Remington's Pharmaceutical Sciences, 16^{th} edition, Osol, A. (ed), 1980.

The agent may be administered in a localised manner to the brain or other desired site, e.g. tumour tissue, or it may be delivered systemically in a manner such that it targets the brain or other cells.

Further provided is a composition comprising a cleaved teneurin product and a cellular target of the cleaved teneurin product wherein said target might be without limitation PML, Zic, or ponsin, or any target identified by expression array analysis (Table 1 and 2). The combination of the cleaved teneurin product and its cellular target may be used as a pharmaceutical or for the manufacture of a medicament for the treatment or prophylactic treatment of tumourigenesis or cancer or neurodevelopmental or neuroregenerative disease. The production or administration of such a pharmaceutical for use in the diagnosis, prognosis or therapeutic treatment will be performed as hereinabove described.

### EXAMPLES

The examples are described for the purposes of illustration and are not intended to limit the scope of the invention. Other embodiments of the invention will occur to those skilled in the art in the light of these.

Methods of molecular genetics, protein and peptide biochemistry and immunology referred to but not explicitly described in this disclosure and examples are reported in the scientific literature and are well known to those skilled in the art. For example, standard methods in genetic engineering are carried out essentially as described in Sambrook et al., Molecular Cloning: A laboratory manual, 3rd Ed., Cold Spring Harbor Laboratory Press, Cold Spring Harbor N.Y., 2001 and antibody-related techniques according to: Using antibodies: a laboratory manual/Ed Harlow, David Lane. Cold Spring Harbor, N.Y.: Cold Spring Harbor Laboratory Press, c1999. xiv, 495 p.)

Antibodies and DNA Constructs

The following antibodies and constructs are easily prepared by routine methods and/or are available commercially: anti-teneurin-2 (Rubin et al. (1999) Dev Biol 216: 195-209), anti-VSV (affinity-purified peptide antibody described in Kreis (1986) Embo J 5,931-41), anti-FLAG (M2, Stratagene), anti-GAL4 (DGB RK5C1, Santa Cruz Biotechnology Inc.), anti-PML (PG-M3, Santa Cruz), anti-myc (c-Myc 9E10, Santa Cruz); anti-HA (1 2CA5, Roche); Alexa594- and Alexa488-conjugated goat-anti mouse and goat anti-rabbit IgG (Molecular Probes), horseradish peroxidase coupled anti-mouse and anti-rabbit IgG (Soccochim). An antiserum was raised against the N-terminal part of the cytoplasmic domain of teneurin-1 (aminoacids 1-158; Minet et al. 1999) expressed as a His-tagged protein in E. coli and purified by affinity chromatography on a Nickel column (Quiagen). The IgG fraction of this antiserum was purified over Protein A Sepharose (Amersham) according to standard procedures.

Constructs: pFR-luc (luciferase reporter plasmid; Stratagene), pSV-β-Galactosidase (Promega), pCMV-AD, PCMV-BD and pBD-NFκB (Stratagene), p-CMX-PML and p-CMX-PML-RAR (described in Doucas et al. (1993) Proc Natl Acad Sci USA 90(20):9345-9), pEF-zic1 (described In Aruga et al. (1996) J Biol Chem. 271(2):1043-7), pXP2-APOE1 89 (luciferase reporter plasmid under the control of an apolipoprotein E promoter; described in Salero et al., (2001) J Biol Chem. 276(3):1881-8) are used below.

Teneurin-2 Constructs

Seven different teneurin-2 constructs were used, these are namely BDAD-CTEY, BDAD-CTE, BDAD-CT, BDAD-C, C, TE, TEY. They are named according to the teneurin-2 protein domains contained within their coding regions, namely CTEY (for Cytoplasmic, Transmembrane, EGF-like and YD-repeats), containing the complete coding region of the long form of teneurin-2 including the cytoplasmic domain as well as the entire long form of the extracellular domain (described in Tucker et al. (2001) Dev Dyn 220: 27-39, Accession No. AJ279031); TEY, containing the entire long form of the extracellular domain; CTE, corresponding to a short splice variant of teneurin-2 containing the complete coding region of the short variant of teneurin-2 (Accession No. AJ245711) (described in Rubin et al. (1999) Dev Biol 216: 195-209); or TE, the short splice variant of teneurin-2 but without a cytoplasmic domain. Construct C represents the soluble cytoplasmic domain of teneurin-2. It encodes the first 372 amino acids of the teneurin-2 sequence as described in Rubin et al. 1999 (Rubin et al. (1999) Dev Biol 216: 195-209) followed by a VSV-G tag (an eleven amino acid sequence from residues 501 to 511 of the vesicular stomatitis virus (VSV) glycoprotein) for detection. In four constructs teneurin-2 was coupled to the GAL4 binding domain (BD) and the NFκB activation domain (AD) generating BDAD-teneurin-2 fusion proteins. These constructs were cloned by multiple PCR. The product of the PCRs comprised bases 675-1118 of pCMV-BD coding for the GAL4 binding domain (BD), bases 703-1267 of pCMV-AD coding for the (AD) and bases 1-630 of teneurin-2 coding for the first 210 aminoacids of the cytoplasmic domain of teneurin-2 until the BIpI site. These fragments were connected by the method of SOE (gene splicing by overlap extension, described in Horton R. M. (1995) Mol. Biotechnol., 3(2); 93-99) and the resulting construct was then cloned into the BamHI/Blpl site of the preexisting pcDNA3/Neo vectors (Invitrogen) containing teneurin-2 constructs of different lengths as described above.

Transient Transfections

HT1 080 human fibrosarcoma (ATCC CRL-12012) and COS-7 cells (SV40-transformed kidney fibroblasts from the African green monkey, ATCC CRL-1651) were routinely maintained In DMEM medium (Dulbecco's Modified Eagles Medium) supplemented with 10% FCS (fetal calf serum). For transient transfections the cells were seeded in 6-well plates or 4-well staining dishes (Greiner). Twelve hours later they were transfected with the expression vectors containing the constructs (1 μg of each vector) described above and in Example 2 (Zic), Example 4 and Example 5 by using FUGENE-6 (3 μl, 6 μl or 9 μl for one, two to three, or four different plasmids, respectively). The cells were treated with the following substances at least five hours after transfection: ALLN (25 μg/ml; N-acetyl-leu-leu-norleu-AL; Sigma); tunicamycin (2 μg/ml; Sigma) or lactacystin (10 μM; e.g. Sigma) for four or eight hours prior to harvesting. Twenty-four hours after transfection the cells were rinsed in PBS (Phosphate-buffered saline) and processed for either measuring luciferase and β-galactosidase activities, western blotting or immunofluorescence, essentially as described below.

Stable Cell Lines

Construct C was subcloned into the ecdyson-inducible expression vector pIND (Invitrogen) and transfected into EcR 293 cells according to the manufacturer's manual (Invitrogen) (EcR cell lines have been stably transformed with the regulatory vector, pVgRXR, for use in the Ecdysone-lnducible Mammalian Expression System. The cell lines express the ecdysone receptor, which regulates muristerone-dependent induction from pIND and pIND(SP1). Clones were tested for the inducible expression of construct C by the addition of increasing concentrations of Ponasterone (1-10 μg/ml; Invitrogen) by immunoblotting using anti-VSV antibodies.

Luciferase and β-Galactosidase Assays

The cells were lysed by adding reporter lysis buffer (Promega). Serial dilutions of the lysed cell suspension were then pipetted into Microlite™ luciferase plates (Dynex Technologies) and the luciferase activity was measured in a Microlumat (LB96P, EG+G Berthold) by automatic injection of luciferin substrate solution (2 mM luciferin, 100 mM ATP in 250 mM glycin pH 7.8, 150 mM MgSO₄). All luciferase activities were normalised with respect to the transfection efficiency by co-transfecting a β-galactosidase vector. To determine β-galactosidase activity the diluted cell suspensions were incubated with the substrate solution (4.5 mM 2-nitrophenyl-β-D-galactopyranoside in 0.2 M Na-phosphate, 2 mM MgCl₂, 0.1 mM β-mercaptoethanol) for 30 min at 37° C. To stop the enzymatic reaction, three times the sample volume of 1 M Na₂CO₃ was added and the OD was measured at 405 nm in a microplate reader (BioRad).

Western Blotting

Teneurin-2 constructs C, CT and CTE were extracted by adding SDS sample buffer containing 20% β-mercaptoethanol directly to the cells (100 μl/3.5 cm plate). Extraction of the nuclear constructs BDAD and BDAD-C was achieved by performing nuclear fractionation. The transfected cells were harvested by scraping off the cell layer in 200p1 lysis buffer (10 mM HEPES pH 7.5, 0.5% triton X-1 00, 300 mM sucrose, 100 mM NaCl, 2 mM MgCl₂, protease inhibitors (Complete™, Roche Diagnostics) on ice and subsequent centrifugation for 10 min at 2000 rpm in an Eppendorf centrifuge. The resulting pellet was resuspended in lysis buffer and centrifuged again. The final pellet was then dissolved in SDS sample buffer containing 20% β-mercaptoethanol, 6 M urea and protease inhibitors (Complete™). Before loading on an 8% SDS-PAGE gel (sodium dodecyl sulfate-polyacrylamide gel electrophoresis), DTT was added to a final concentration of 10 mM.

The transmembrane constructs BDAD-CTE and BDAD-CTEY were extracted from the cells by the following procedure. The cells were extracted on ice by 200 μl of a hypotonic buffer (2 mM Na-phosphate buffer pH 7.5, 20 mM KCl, 1 mM β-mercaptoethanol), scraped off and centrifuged for 10 min at 8000 rpm at 4° C. in an Eppendorf centrifuge. The resulting pellet was reconstituted in 100 μl of detergent buffer (50 mM Tris pH 8, 1% NP40, 150 mM NaCl, 5 mM EDTA, 6 M urea, protease inhibitors (Complete™), incubated for 20 min at 37° C. and centrifuged for 10 min at 13'000 rpm. An equal volume of SDS sample buffer containing 20% β-mercaptoethanol, 6 M urea and protease inhibitors (Complete™) was added to the supernatant and incubated for 1 hour at 52° C. After DTT was added (10 mM), the samples were loaded on a 6% SDS-PAGE gel.

The gels were transferred to PVDF (polyvinylidene fluoride) membranes. The proteins were detected by anti-GAL4 antibody (BDAD and BDAD-C) or by anti-teneurin-2 serum (BDAD-CTE and BDAD-CTEY), horseradish peroxidase coupled secondary antibodies and ECL SuperSignal® (Pierce).

Immunofluorescence

The cells grown on 4-well staining dishes (Greiner) were fixed with 4% PFA for 30 min at room temperature, and permeabilised with 0.1% Triton X-100 for 5 min. Incubation with primary antibodies was performed for 60 min and that with secondary antibodies for 30 min both at room temperature whereas the cells were washed in PBS after each incubation. Finally the specimens were mounted in Moviol (Hoechst, Frankfurt Germany) and examined and photographed using a Zeiss Axiophot microscope (Carl Zeiss Ud.) connected to a 3CCD camera (Sony).

Microarray Analysis

Microarray analysis was performed using Affymetrix HG-U133A GeneChips™ (Affymetrix, Santa Clara, USA). 10 μg of total RNA (isolated from EcR-293 cells (Invitrogen)) was reverse transcribed using the SuperScript Choice system for cDNA synthesis (Life Technologies) according to the protocol recommended by Affymetrix (GeneChip Expression Analysis: Technical Manual (2001) p. 2.1.14-2.1.16). The oligonucleotide used for priming was 5′-ggccagtgaattgtaatacgactcactatagggaggcgg-(t)₂₄-3′ (SEQ ID NO: 9). Double-stranded cDNA was cleaned by phenol:chloroform extraction and the aqueous phase removed by centrifugation through Phase-lock Gel (Eppendorf). In vitro transcription was performed on 1 μg of cDNA using the Enzo BioArray High Yield RNA transcript labelling kit (Enzo Diagnostics, USA) following the manufacturer's protocol. The cRNA was cleaned using RNAeasy clean-up columns (Qiagen). To improve the recovery from the columns the elution water was spun into the matrix at 27 g and then left for one minute prior to the standard 8000 g centrifugation recommended by Qiagen. This low speed wetting step gave nearly double the yield of eluted RNA. The cRNA was fragmented by heating in 1× fragmentation buffer (40 mM Tris-acetate pH 8.1, 100 mM KOAc, 30 mM MgOAc). 10 μg of fragmented cRNA were hybridised to a HG-U133A GeneChip (Affymetrix) using their standard procedure (45° C., 16 hours). Washing and staining was performed in a Fluidics Station 400 (Affymetrix) using the protocol EukGE-WS2v4 and scanned in an Affymetrix GeneChip scanner. Chip analysis was performed using the Affymetrix Microarray Suite v5 (target intensity 500 used for chip scaling) and GeneSpring 4.2.1 (Silicon Genetics). Changes In gene expression were assessed by looking for concordant changes between replicates using a signed Wilcoxon rank test (as recommended by Affymetrix). The “change” p-value threshold was <0.003 for increase and >0.997 for decrease. After concordance analysis these values become <9×10⁻⁶ and >0.999991 respectively. Any gene whose detection p-value was >0.05 in all experimental conditions was discarded from the analysis as being unreliable data.

### Example 1

### Localisation of the Teneurin-2 Cytoplasmic Domain to the Nucleus and Its Co-Locallization with PML

This Example demonstrates that teneurin co-localizes with PML (promyelocytic leukaemia protein) in nuclear bodies. It was found that the cytoplasmic domain of teneurin-2 (referred to as construct C) is translocated to the nucleus if transfected into human HT1080 fibrosarcoma cells. Transfection of HT1080 cells with construct C led to accumulation of the cytoplasmic domain in the nucleus, where its presence was confined to discrete spots within the nucleus. In contrast, the immunofluorescence staining pattern obtained with the transmembrane version of teneurin-2 (construct TEY) showed accumulation of the protein on the cell surface.

The nuclear localisation reflected a very similar punctuate pattern obtained by staining for PML protein (reviewed in Seeler et al. (1999) Curr Opin Genet Dev. 9(3):362-7; or Salomoni and Pandolfi (2002) Cell 108,165-70) and is thus very suggestive of nuclear bodies termed PODs (promyelocytic oncogenic domains) or PML bodies. Double-immunofluorescent stainings of C transfected cells showed substantial overlap of PML staining with that of the cytoplasmic domain of teneurin-2.

Since PML bodies are involved in a number of functions associated with transcriptional control (reviewed in Zhong et al. (2000) Nat Cell Biol. 2(5):E85-90), the colocalisation of teneurin-2 and PML in nuclear bodies was further investigated by cotransfecting C with PML, or C with PML-RAR (PML—retinoic acid receptor). Transfection of PML into cells containing endogenous PML protein leads to a massive enlargement of the nuclear bodies (Doucas et al. (1996) Biochim Biophys Acta. 1288(3):M25-9), whereas transfection of PML-RAR results in the destruction of the PML body architecture (Mu et al. (1994) Mol Cell Biol. 14(10):6858-67). The present inventors also observed these effects. An equal staining pattern was detected after cotransfection of PML and C, where teneurin-2C (i.e. the cytoplasmic domain) was found in the enlarged PML bodies. Furthermore destruction of the PML body architecture after transfection with PML-RAR also changed the staining pattern with co-transfected C, which was no longer accumulated in discrete spots in the nucleus but seemed to be expressed homogeneously throughout the cells. These results suggest the presence of teneurin-2 C within nuclear bodies thus supporting a putative transcriptional regulatory function of the cytoplasmic domain of teneurin-2.

### Example 2

### Teneurin-2 Inhibits Zic Transcriptional Regulatory Activity

In this Example, it was determined whether the zinc finger transcription factor Zic, a vertebrate homologue of opa would influence, or be influenced by, the cytoplasmic domain of teneurin-2. When both proteins, the cytoplasmic domain of teneurin-2 and Zic, were expressed in HT1080 cells by transient transfections, a marked down-regulation (over ten-fold) of teneurin-2 C was observed compared to its usual expression level. In contrast, co-transfection of the two constructs had no effect on the Zic level. This effect was specific for teneurin-2 since BDAD was not down-regulated by the presence of Zic in an analogous analysis. The Zic induced downregulation of teneurin-2 C was counteracted by the addition of the proteasome inhibitor lactacystin. By immunofluorescence staining of the transfected cells we observed that Zic-transfected cells revealed a relatively diffuse nuclear staining of Zic and in nuclei containing high amounts of Zic protein, the punctuate staining of teneurin-2 C was not seen.

In order to examine a potential effect of the teneurin-2 C on the transcriptional activity of Zic, EcR 293 cells stable transfectants were produced. In these cells teneurin-2 C was only expressed upon addition of ponasterone, which induces expression of the cytoplasmic domain of teneurin-2. These cells were transiently transfected with Zic and the ApoE-luciferase reporter construct known to be activated by Zic. The presence of Zic led to a dramatic increase in luciferase acitivity. After the induction of teneurin-2 C by ponasterone a marked reduction in the expression level of the reporter gene was observed. These results suggested that the cytoplasmic domain of teneurin-2 has an inhibitory effect on the transcriptional activity of Zic and this effect is more pronounced in the presence of ALLN which was shown to stabilize teneurin-2 C.

### Example 3

### Functional Interaction of Teneurin-1 with Ponsin

Yeast two hybrid screens using the cytoplasmic domains of teneurin-1 and -2 as bait were carried out to determine if teneurins interact with other proteins. The screen was performed with the DupLEX-A™ system (OriGene Technolgies, Inc.) according to their User's Manual (Version 2.9 8/98). First, the entire cytoplasmic domains (aminoacids 1-300 of teneurin-1; Minet et al. 1999 and aminoacids 1-376 of teneurin-2; Rubin et al. 1999) were used. However, both of these constructs resulted in self-activation of the LexA-dependent target genes and thus could not be used to search for interaction partners. We next split each of the cytoplasmic domains in two parts Ten-1 a, Ten-1 b and Ten-2a, Ten-2b, respectively (Ten-1a represents aminoacids 1-155 and Ten1b 156-300 of teneurin-1 and Ten-2a represents aminoacids 1-168 and Ten2b 169-376 of teneurin-2). Both, Ten-1a and Ten-2a were still self-activating the LeXA-dependent genes, implying a possible function for teneurins in transcriptional control. Ten-1b and Ten-2b could be used to screen a whole mouse 19day embryo DupLex-A cDNA library (OriGene). The screen with Ten-1b resulted in an interacting cDNA clone that encoded the third SH3 domain of a protein called ponsin (Mandai et al. 1999).

The binding of full length ponsin to the cytoplasmic domain of teneurin-1 was confirmed by co-transfection and co-immunoprecipitation of a myc-tagged posing expression plasmid (ponsin-2 in pCMV5-Myc as described in Mandai et al. 1999) and the full length cytoplasmic domain of teneurin-1 (aminoacids 1-300; Minet et al. 1999) in Cos-7 cells. Cultures of Cos-7 cells (3.5 cm dishes at 70% confluency) were co-transfected with 1 μg of each DNA using fugene (Roche). 48 hours later cell layers were washed with PBS and the cell layers frozen on dry ice to break up cells, which were then scraped into 300 μl of PBS including proteinase inhibitors (Complete proteinase inhibitor cocktail tablets, Roche). This cell extract was centrifuged at 14'000 rpm. To the supernatant 150 μl of RIPA buffer (150 mmM NaCl; 50 mM Tris-HCl pH 8.0; 1% NP-40; 0.5% Deoxycholic acid; 0.1% SDS; 50 mM NaF; 0.5 mM Na₃VO₄) was added followed by immunoprecipitation with 1 μg of anti-teneurin-1 IgG. The cell lysate was incubated with the anti-Ten-1 antibody for 90 minutes slowly rotating at 4° C. Then 30 μl of protein A Sepharose beads (4 Fast Flow; Amersham) washed in RIPA buffer were added. After 60 minutes of rotation at 4° C. the beads were collected by centrifugation and washed three times with PBS. The beads were eluted by boiling in 50 μl of SDS-PAGE sample buffer and analyzed by western blotting. The eluate was run in two parallel lanes on a 10% SDS-PAGE and blotted to PVDF membranes. The immunoprecipitated teneurin-1 was detected with an anti-FLAG antibody and the co-precipitated ponsin by anti-myc followed by secondary antibodies and detection using a chemiluminescence kit (ECL, Amersham). Whereas immunoprecipitated teneurin-1 contained ponsin, precipitates with control IgG did not contain any detectable ponsin.

The interaction of teneurin-1 with ponsin was further confirmed by immunohistochemistry of co-transfected cells. Transfection of ponsin alone results in a cytoplasmic and cytoskeleton-associated expression pattern, whereas the soluble cytoplasmic domain of teneurin-1 accumulates in the nucleus. Upon co-transfection of the cytoplasmic domain of teneurin-1 and ponsin, ponsin translocates together with teneurin-1 into the nucleus. Thus teneurin-1 co-localizes with and binds to ponsin, which means that they will influence each others function in the regulation of cell adhesion, cytoskeleton assembly and possibly transcription.

### Example 4

### Morphological Changes of Cells Expressing the Cytoplasmic Domain of Teneurin-2

Cell extracts from COS-7 green monkey kidney fibroblasts were prepared 24 hours after transfection with CTEY, TEY, CTE and TE constructs and analyzed by SDS-PAGE and immunoblotting with anti-teneurin-2 serum. Each construct resulted in the presence of a major band of roughly the expected size of 280 kD, 240 kD, 120 kd and 80 kd, respectively. The proteins without the cytoplasmic tails (TEY and TE) were about 40kd smaller than the corresponding proteins with these domains present (CTEY and CTE). Immunostaining of the transfected cells without permeabilization prior to antiserum incubation revealed the presence of the extracellular domains of teneurin-2 on the cell surface. The morphology of the cells expressing the constructs including the cytoplasmic domains was very different from the ones without the cytoplasmic domain. Whereas CTEY and CTE induced prominent filopodia in the transfected cells, TEY and TE was present in cells with smooth surfaces. This implies an interaction of the teneurin-2 cytoplasmic domain with cytoskeletal components.

### Example 5

### Homophilic Binding of Teneurin Induces Cleavage

To isolate stable cell lines expressing teneurin-2 constructs human HT1080 fibrosarcoma cells were transfected and replated to allow clonal growth of the transfected cells. Several clones representing the constructs were screened for recombinant protein expression by immunofluorescence as well as by immunoblotting. Phaloidin-staining and detection of the recombinantly expressed teneurln-2 proteins with the anti-teneurin-2 antibody by immunohistochemistry was done as follows:

24 to 72 hours after transfection the cells were rinsed once with CA²⁺- and Mg²⁺-free PBS (PBS CMF) and either fixed with 4% formaldehyde in PBS CMF for immunohistochemistry or scraped off the dish and solubilized in SDS-PAGE sample buffer for Western blot analysis. To permeabilize the fixed cells 0.2% Triton X-100 in PBS CMF was applied for 15 min. following this step the permeabilized cells, like the intact cells, were rinsed with PBS CMF and incubated in blocking buffer (3% BSA in PBS CMF) for 15 min. The primary antibodies (anti-teneurin-2, anti-VSV tag, as well as the fluorescein-coupled secondary antibody (Jackson ImmunoResearch Laboratories, Inc., West Grove, Pa.) were diluted in the blocking buffer. Along with the secondary antibody phalloidin coupled to rhodamine was incubated (Sigma, Buchs, Switzerland). After each of the incubations, cells were rinsed In PBS CMF. For viewing, cells were mounted in Moviol (Calbiochem, La Jolla, Calif.). For Western blot analyses samples were separated on a 10% SDS-PAGE, transferred onto a nylon membrane, and treated as described by Hagios et al. (1996) J Cell Biol. 134(6):1499-512.

Teneurin-2 containing cellular extracts for immunoblotting were prepared as follows: After washing the cells on 5 cm culture dishes with cold PBS, plates were frozen at minus 20° C. After thawing, they were extracted for 30 min on ice with 600 μl of hypotonic buffer (20 mMKCl, 2 mM Na-phosphate pH 7.0/1 mM β-mercaptoethanol). Cells were collected by scraping with a rubber policeman and transferred into Eppendorf tubes. After spinning for 10 minutes at maximum speed the pellet was dissolved at 37° C. for 20 min in 60 μl detergent buffer (150 mM NCl/50 mMTris pH8/1% NP-40/6M urea, 5 mM EDTA) per plate. An equal volume of SDS-PAGE sample buffer (0.2M Tris-HCl pH6.8/4%SDS/17.4% glycerol/20% β-mercaptoethanol/6M urea) was added and the sample was incubated for 1 hour at 52° C. The samples were separated on SDS-PAGE and transferred to PVDF membranes. The teneurins were detected by the anti-teneurin antiserum (Rubin et al. (1999) Dev Biol. 216(1):195-209) and the signals revealed using the ECL western blotting system (Amersham).

We were able to select several clones of the two constructs, TEY and TE, that lack the cytoplasmic domain. The clones expressing the long form of the extracellular domain (TEY) showed a much flatter morphology than either the parental cells (HT1080) or the cells expressing the short teneurin extracellular domain (TE) as can be seen both on the phase contrast pictures as well as after phalloidin staining of the actin cytoskeleton. Despite their flat morphology the clones of TEY did not contain a more pronounced actin cytoskeleton than the parental cells and all cells revealed mostly cortical actin staining.

In addition to their flat phenotype we noticed that the cell clones of TEY grew in epithelial cell-like patches suggestive of increased cell-cell adhesion, whereas the parental cells and clone TE grew as dispersed fibroblast-like cells. Therefore, we investigated whether or not these clones showed increased cell-cell adhesion in an aggregation assay of cells In suspension. Cells were harvested by incubation with 0.2% EDTA, pelleted by centrifugation and resuspended at 2.5×10⁵ cells/ml of Leibovitz L15 medium (Gibco) containing 1% fetal calf serum (Gibco). Three milliliters of each cell suspension were added to 5 cm bacterial plates (Sterilin) and incubated at 37° C. on a rotary shaker at 80 rpm. Photographs were taken at 15 min intervals and cell aggregation was analyzed by counting single cells versus cells in double, triple or higher number aggregates. The aggregation index was calculated as Nₒ-Nₜ/Nₒ, where Nₒ is the initial number of particles corresponding to the total number of cells, and Nₜ is the number of remaining particles at the incubation time point t. While the parental HT1080 cells as well as the cells containing the TE construct remained as single cells, all clones expressing the long teneurin-2 extracellular domain (TEY) aggregated into clumps of cells. The presence of the extracellular domain of teneurin-2 on these cells resulted in their homophilic aggregation. Quantitative comparison of the cell aggregation showed that after 30 minutes of incubation all TEY cell lines had already approached maximal aggregation values, whereas the HT1080 and TE cells did not aggregate even after 90 minutes of incubation.

Further the effect of the expression of the teneurin-2 constructs on Nb2a neuroblastoma cells was investigated. As reported in Rubin et al. (1999) Dev Biol. 216(1):1 95-209, expression of CTE constructs in Nb2a cells led to the induction of filopodia and enlarged growth cones. This effect was not seen after transfection of the TE construct lacking the cytoplasmic domain and the protein mainly localized to cell bodies and not to neurites. This suggests that the cytoplasmic domain is required for the translocation of the teneurin protein to neurites and growth cones. The transfection of the longer constructs CTEY and TEY resulted in quite different teneurin-2 expression patterns. Both of these proteins were expressed on the cell surfaces and were heavily enriched in cell-cell contact areas giving further support for the promotion of homophilic interactions between these neuronal cells. The CTEY expression led in addition to the accumulation of actin to these teneurin-rich cell-cell contacts as revealed by phalloidin staining, which was not the case for the TEY construct. This provides further support for an interaction between teneurin-2 and the actin cytoskeleton through its cytoplasmic domain.

### Example 6

### Cleavage and Stabilization of the Cytoplasmic Domain

The nuclear localization of the cytoplasmic domain and its transcriptional regulatory role would mean that the wild-type transmembrane teneurin-2 would have to be specifically cleaved in or at the plasma membrane possibly upon ligand binding. The cytoplasmic domain would thereby be released and translocated to the nucleus. To establish this process, fusion proteins of full length teneurin-2 or smaller fragments comprising the transmembrane domain, attached to a GAL4 DNA binding domain (BD) and a NFκB activation domain (AD) were introduced Into HT-1080 and Cos-7 cells. Cleavage of the fusion protein and translocation of BDAD-C to the nucleus can be detected by binding of BD to specific GAL4 binding sequences on a co-transfected luciferase reporter plasmid, which results in the subsequent initiation of luciferase gene expression through AD.

BDAD and BDAD-C serving as positive controls In this experiment were detectable on a Western blot of nuclear extract by anti-GAL4 antibodies. In addition, BDAD-C accumulation in the nucleus was confirmed by immunofluorescent staining of permeabilised cells. At the same time BDAD-CTE and BDAD-CTEY could be identified as part of the (plasma) membrane by western blot of a membranous cell fraction and by immunofluorescence of non-permeabilised cells.

For analysis of the luciferase activity induced by the teneurin-2 fusion constructs, HT1080 cells were cotransfected with the respective BDAD-teneurin-2 constructs, the luciferase reporter plasmid as well as a β-Galactosidase construct for normalisation of transfection efficiencies. BDAD-CTE, BDAD-CT and BDAD-C led to an induction of luciferase activity over the negative control (BD construct). However, BDAD-CTEY being the largest fusion protein did not lead to a significantly enhanced activity. Cleavage of the shorter constructs might be constitutive while cleavage of the full length construct might have to be specifically induced, for example by ligand binding.

As described in Example 5, teneurin-2 has been shown to bind homophilically by its extracellular domain and may induce cleavage and translocation of the cytoplasmic domain of the BDAD-teneurin-2 fusion proteins. To test whether homophilic interaction of teneurin-2 represents a signal for cleavage of its cytoplasmic domain, BDAD-CTEY and as control BDAD-CT, were transfected into HT-1 080 cell clones that constitutively express TEY or TE on their surface (described in Example 5 hereinabove) and the luciferase activity produced from the co-transfected reporter plasmid analysed. The luciferase activities obtained were normalized to the transfection of the same constructs into wild type HT1080 cells. Luciferase activity obtained after transfection of BDAD-CTEY into the TEY cells was approximately 6-fold higher than after transfection of BDAD-CT while in TE cells the activity was not affected by the type of construct used for transfection. Thus, the C-terminal of the extracellular domain is required to induce the cleavage of the cytoplasmic domain of teneurin-2.

In summary, the activity of the luciferase reporter gene originates from the cleavage of the BDAD-teneurin-2 fusion proteins at (or in the vicinity of) the membrane because it could be specifically upregulated by homophilic binding of full length but not shorter truncated versions of teneurin-2 extracellular domains on the surface of the cell. BDAD attached to full length teneurin-2 (BDAD-CTEY) led to a significant induction of the luciferase gene only when processing was upregulated by homophilic binding of the extracellular C-terminal part of teneurin-2. In addition, this activation was not observed with the truncated teneurin-2 fusions described above. Nevertheless, the shorter counterparts seemed to be cleaved without stimulation. Although not wishing to be bound by theory, the large BDAD-CTEY construct is expressed less efficiently than its shorter counterparts and the cleavage products might be down-regulated quickly in proteasomes. The induction of luciferase activity following transfection of BDAD-CTEY could indeed be markedly upregulated by the addition of protease inhibitors such as ALLN and lactacystin. Similarly, CTE protein levels also increased as demonstrated by western blot. The addition of ALLN led to the stabilisation of two particular cleavage products, one of which corresponded to the size of the cytoplasmic domain alone.

### Example 7

### Identification of Genes Regulated by Teneurin Signalling

The cDNA encoding the cytoplasmic domain of teneurin-2 containing a C-terminal VSV-tag was cloned into the vector plnd (Invitrogen). EcR-293 cells (Invitrogen) containing the stably integrated plasmid pVgRXR (Invitrogen) were transfected with the plnd teneurin-2 plasmid encoding the cytoplasmic domain. Stable clones were selected and tested for the expression of the cytoplasmic domain of teneurin-2 after addition of ponasterone (Invitrogen). Cells of one positive clone termed K3 was used for the further analysis. Parental EcR-293 cells and K3 cells were grown to 70% confluency before 10 μM ponasterone was added to both cell cultures for 24 hours. Parallel cultures were kept without the addition of ponasterone. After 24 hours mRNA was isolated using Trizol reagent (Gibco) from four different cultures: K3−ponasterone, K3+ponasterone, ECR293−ponasterone and ECR293+ponasterone. This procedure was typically performed in duplicates, resulting in 8 mRNA batches. Each of these batches of mRNA was used to make probes to hybridyze to Affymetrix chips (U133) containing oligonucleotides for the detection of 20'000 potential transcripts. Lists were derived that contained genes that were differentially expressed depending on the presence of ponasterone. Genes that were common to both lists of the two indepently performed duplicates were investigated further and made up the entries of the four lists K3 up, K3 down (genes up- or down-regulated by ponasterone in K3 cells), EcR-293 up, EcR-293 down (genes up- or down-regulated by ponasterone in EcR-293 cells. To exclude any genes that were affected by ponasterone directly, any entries common to the lists of the ECR293 and K3 cells were discarded resulting in the final lists of genes up-regulated (Table 1) or down-regulated (Table 2) by the presence of the cytoplasmic domain of teneurin-2. Table 1 shows the genes up-regulated by the cytoplasmic domain and the ID number of the respective database assigned by Affymetrix. Table 2 shows the genes down-regulated by the cytoplasmic domain and the ID number of the respective database assigned by Affymetrix. Among the genes up- and down- regulated by teneurin-2 many are known to play a role in the regulation of cell growth, differentiation and apoptosis. A group of genes up-regulated by teneurin-2 correlates with genes known to be targets of p53 (Nr. 7, 23, 33, 51, 65 in Table 1). Another group of down-regulated genes corresponded to genes known to play a role in neuronal differentiation and synapse formation and function (Nr. 4, 5, 11, 12, 27, 28, 29, 30, 34, 35, 40, 56, 90 of Table 1). A group of genes down-regulated by teneurin-2 correlated with genes known to be glutathionylated and involved in the regulation of the redox status of cells (Nr. 3, 4, 11, 44, 46, 69 of Table 2). Another group of down-regulated genes corresponded to genes known to be targets of myc (Nr. 1, 3, 4, 11, 14, 31, 69 of Table 2).

### Example 8

### Proliferation Assay

The effect of the expression of the cytoplasmic domain of teneurin-2 on cell proliferation was tested. K3 cells (EcR-293 cells (Invitrogen) containing the stably integrated plasmid pVgRXR (Invitrogen) and the plnd teneurin-2 plasmid encoding the cytoplasmic domain, described in Example 7) were cultured in the presence or absence of ponasterone, which induces expression of the cytoplasmic domain of teneurin-2. Typically, over a 5 day period, parallel cultures were fixed with 4% formaldehyde every day and cell numbers were determined by staining the cells with crystal violet (0.1% in H₂0). In the presence of ponasterone and thus in the presence of the cytoplasmic domain of teneurin, the K3 cells were retarded in their growth rate to about 50% of the untreated cells. Proliferation of EcR-293 cells without transfected cytoplasmic domain of teneurin-2 was not affected by the addition of ponasterone, proving that the growth retardation was specifically dependent on the presence of the cytoplasmic domain of teneurin-2.

### Example 9

### The Cytoplasmic Domain of Teneurin-1 Can Be Detected in Nuclei of C. elegans Embryo Cells

Ten-1 is the C. elegans orthologue of the Drosophila pair-rule gene ten-m. We describe here the gene structure of ten-1 encompassing two promoters that control the expression of two different ten-1 transcripts. This results in the expression of two type II transmembrane protein variants differing In their cytoplasmic domains. The 5′ ends of the ten-1 cDNAs were determined by RT-PCR using splice leader 1 (SL1) as the 5′ primer and a ten-1 specific oligonucleotide as the 3′ primer. cDNA fragments were prepared by RT-PCR using Superscript RNaseH(−) reverse transcriptase (Life Technologies). 5′ cDNA ends were amplified with Expand HiFi polymerase (Roche) using SLi as a 5′ primer and the gene specific primer AGCACGTGTCGCTATCGTCG (SEQ ID NO: 10) using cDNA prepared from mixed stage worms. The RT-PCR reaction using mixed stage mRNA as template resulted in two products differing in size. By sequencing these bands two different cDNA species could be identified. One of them corresponded with a minor difference at the 5′ end to the transcription start of the predicted open reading frame R16F6.4 and the other one contained in addition the predicted open reading frame F28F5.1 together with a newly discovered exon. Thus the ten-1 gene is under the control of alternative promoters resulting in two different transcripts that encode two Ten-1 proteins differing in their N-terminal sequences. The long form contains N-terminal to a predicted transmembrane sequence a cytoplasmic domain of 218 aa whereas the short form has only 37 aa that are in common between both variants. The intracellular sequences do not contain any predicted domains but contain a consensus sequence for tyrosine phosphorylation. The long form harbors a proline-rich stretch at the N-terminus and a potential bipartite nuclear localization signal. The N-terminal cytoplasmic domains are followed by a transmembrane domain making Ten-1 a type II transmembrane protein.

For DNA constructions 4 Kb of the upstream promoter where amplified with the primers CATTGGTCMTTGGCGCGCCCATTCGCAGACG (SEQ ID NO: 11) and ATTAGGCGGTGGGGGTACCGCATTCG (SEQ ID NO: 12) and cloned into the Ascl/Kpnl sites of pPD117.28 (A. Fire). 3 Kb of the downstream promoter where amplified with the primers GMTTCGCATGCAAATGTGMGCATG (SEQ ID NO: 13) and CCACCAGGTACCGGATCACCATTGTTC (SEQ ID NO: 14) and cloned into the Sphl/Kpnl sites of pPD1 17.28 (A. Fire). DNA encoding the long intracellular domain was amplified with the primers CAGAGTGCGGCCGCCCGTGCGTTTCG (SEQ ID NO: 15) and GGCTAGGMTTCATTCCATTTGGATGG (SEQ ID NO: 16). DNA encoding the short intracellular domain was amplified with the primers TTACAATTTTTCAGGCGGCCGCAAGTTGGC (SEQ ID NO: 17) and GGCTAGGAATTCATTCCATTTGGATGG (SEQ ID NO: 18).

For antibody production, anti-Ten-1 antibodies ware raised against the Ten-1 specific peptides from the N-terminus of the long variant (MFQHRTTNAQGPPPNRPMPR) (SEQ ID NO: 19) and the common C-terminus (PAHQSGLLASVHSWKFRKSE) (SEQ ID NO: 20). The peptides where synthesized and the rabbits immunized at Neosystem (Strasburg, France). All sera were affinity purified using the respective peptides coupled to CNBr-activated Sepharose 4B columns according to standard procedures.

Antibodies against the N-terminus of the long ten-1 variant (anti-N) and the one against the C-terminus of the protein (anti-C) were used to stain early embryos of C. elegans. Anti-C stained all membranes while anti-N stained in addition also the nuclei of the Ten-1 expressing cells. This was also prevalent in the cells lining the gut where anti-C stained the cell membranes and anti-N stained the predominantly the nuclei. Although not wishing to be bound by theory, the endogenous Ten-1 might normally be proteolytically processed and the translocation of the cytoplasmic domain could be a physiological process in Ten-1 signaling. The nuclear translocation of the long cytoplasmic domain of Ten-1 can be confirmed by overexpression of both cytoplasmic domains as GFP fusion proteins under a heat shock promoter. Transient expression of these constructs revealed that the long cytoplasmic domain accumulated in the nuclei of the cells whereas the short form remained cytoplasmic.

Ten-1 was characterized as a novel transmembrane protein of C. elegans and is required for gametogenesis, early embryogenesis, and hypodermal cell migration. In later stages of development it is involved in neuronal migration and pathfinding, distal tip cell migration and establishing of the somatic gonad. Furthermore, it is also required for pharynx and gut development as well as for proper defecation. Although not wishing to be bound by theory, Ten-1 might act as a receptor for morphogenetic cues and it directly signals to the nucleus by proteolytic release of its cytoplasmic domain from the cell membrane and by translocation to the nucleus.

TABLE 1

| | | | Locus | Ref Seq | |
|---|---|---|---|---|---|
| | | Unigene | Link | (NM_)/Gene | |
| Nr. | Affimetrix ID | ID | ID | Bank ID | Ref Seq ID + Annotation |
| 1 | 200620_at | Hs.11441 | 9528 | NM_004872.1 | NM_004872 chromosome 1 open |
| | | | | | reading frame 8 |
| 2 | 200710_at | Hs.82208 | 37 | NM_000018.1 | NM_000018 acyl-Coenzyme A |
| | | | | | dehydrogenase, very long chain |
| | | | | | precursor |
| 3 | 200804_at | Hs.74637 | 7009 | NM_003217.1 | NM_003217 testis enhanced gene |
| | | | | | transcript (BAX inhibitor 1) |
| 4 | 200848_at | Hs.4113 | 10768 | AA479488 | NM_006621 S- |
| | | | | | adenosylhomocysteine hydrolase- |
| | | | | | like 1 |
| 5 | 200849_s_at | Hs.4113 | 10768 | AA479488 | NM_006621 S- |
| | | | | | adenosylhomocysteine hydrolase- |
| | | | | | like 1 |
| 6 | 201057_s_at | Hs.7844 | 2804 | NM_004487.1 | NM_004487 golgi autoantigen, golgin |
| | | | | | subfamily b, macrogolgin (with |
| | | | | | transmembrane signal), |
| 7 | 201236_s_at | Hs.75462 | 7832 | NM_006763.1 | NM_006763 BTG family, member 2 |
| 8 | 201301_s_at | Hs.77840 | 307 | BC000182.1 | NM_001153 annexin IV |
| 9 | 201302_at | Hs.77840 | 307 | NM_001153.2 | NM_001153 annexin IV |
| 10 | 201494_at | Hs.75693 | 5547 | NM_005040.1 | NM_005040 prolylcarboxypeptidase |
| | | | | | (angiotensinase C) |
| 11 | 201565_s_at | Hs.180919 | 3398 | NM_002166.1 | NM_002166 inhibitor of DNA binding |
| | | | | | 2, dominant negative helix-loop-helix |
| | | | | | protein |
| 12 | 201566_x_at | Hs.180919 | 3398 | D13891.1 | NM_002166 inhibitor of DNA binding |
| | | | | | 2, dominant negative helix-loop-helix |
| | | | | | protein |
| 13 | 201637_s_at | Hs.82712 | 8087 | NM_005087.1 | NM_005087 fragile X mental |
| | | | | | retardation-related protein 1 |
| 14 | 201663_s_at | Hs.50758 | 10051 | NM_005496.1 | NM_005496 SMC4 structural |
| | | | | | maintenance of chromosomes 4-like 1 |
| 15 | 201813_s_at | Hs.115740 | 9779 | NM_014744.1 | NM_014744 KIAA0210 gene product |
| 16 | 201814_at | Hs.115740 | 9779 | NM_014744.1 | NM_014744 KIAA0210 gene product |
| 17 | 201876_at | Hs.169857 | 5445 | NM_000305.1 | NM_000305 paraoxonase 2 |
| 18 | 201964_at | Hs.154919 | 23064 | N64643 | NM_015046 KIAA0625 protein |
| 19 | 201965_s_at | Hs.154919 | 23064 | NM_015046.1 | NM_015046 KIAA0625 protein |
| 20 | 202436_s_at | Hs.154654 | 1545 | NM_000104.2 | NM_000104 cytochrome P450, |
| | | | | | subfamily I (dioxin-inducible), |
| | | | | | polypeptide 1 |
| 21 | 202437_s_at | Hs.154654 | 1545 | NM_000104.2 | NM_000104 cytochrome P450, |
| | | | | | subfamily I (dioxin-Inducible), |
| | | | | | polypeptide 1 |
| 22 | 203232_s_at | Hs.74520 | 6310 | NM_000332.1 | NM_000332 ataxin 1 |
| 23 | 203409_at | Hs.77602 | 1643 | NM_000107.1 | NM_000107 damage-specific DNA |
| | | | | | binding protein 2 (48 kD) |
| 24 | 203411_s_at | Hs.77886 | 4000 | NM_005572.1 | NM_005572 lamin A/C |
| 25 | 203560_at | Hs.78619 | 8836 | NM_003878.1 | NM_003878 gamma-glutamyl |
| | | | | | hydrolase (conjugase, |
| | | | | | folylpolygammaglutamyl hydrolase) |
| | | | | | precursor |
| 26 | 203594_at | Hs.27076 | 8634 | NM_003729.1 | NM_003729 RTC domain containing 1 |
| 27 | 203789_s_at | Hs.171921 | 10512 | NM_006379.1 | NM_006379 sema domain, |
| | | | | | immunoglobulin domain (Ig), short |
| | | | | | basic domain, secreted, (semaphor |
| 28 | 203810_at | Hs.41693 | 11080 | BG252490 | NM_007034 DnaJ (Hsp40) homolog, |
| | | | | | subfamily B, member 4 |
| 29 | 203999_at | Hs.154679 | 6857 | NM_005639.1 | NM_005639 synaptotagmin I |
| 30 | 205352_at | Hs.78589 | 5274 | NM_005025.1 | NM_005025 protease inhibitor 12 |
| | | | | | (neuroserpin) |
| 31 | 205934_at | Hs.153322 | 5334 | NM_006226.1 | NM_006226 phospholipase C, |
| | | | | | epsilon |
| 32 | 207071_s_at | Hs.154721 | 48 | NM_002197.1 | NM_002197 aconitase 1 |
| 33 | 208478_s_at | Hs.159428 | 581 | NM_004324.1 | NM_004324 BCL2-associated X |
| | | | | | protein, isoform beta NM_138761 |
| | | | | | BCL2-associated X protein, isoform |
| | | | | | alpha NM_38762 BCL2-associated |
| | | | | | X protein, isoform gamma |
| | | | | | NM_138763 BCL2-associated X |
| | | | | | protein, isoform delta NM_138764 |
| | | | | | BCL2-associated X protein, isoform |
| | | | | | epsilon |
| 34 | 208786_s_at | Hs.121849 | 81631 | AF183417.1 | NM_022818 microtubule-associated |
| | | | | | proteins 1A/1B light chain 3 |
| 35 | 208890_s_at | Hs.3989 | 23654 | BC004542.1 | NM_012401 plexin B2 |
| 36 | 208933_s_at | Hs.70333 | 51322 | AI659005 | NM_016628 WW domain-containing |
| | | | | | adapter with a coiled-coil region, |
| | | | | | isoform 1 NM_018604 NM_100264 |
| | | | | | WW domain-containing adapter with |
| | | | | | a coiled-coil region, isoform 2 |
| | | | | | NM_100486 WW domain-containing |
| | | | | | adapter with a coiled-coil region, |
| | | | | | isoform 3 |
| 37 | 208943_s_at | Hs.8146 | 7095 | U93239.1 | NM_003262 translocation protein 1 |
| 38 | 209091_s_at | Hs.136309 | 51100 | AF263293.1 | NM_016009 SH3-containing protein |
| | | | | | SH3GLB1 |
| 39 | 209167_at | Hs.5422 | 2824 | AF016004.1 | |
| 40 | 209238_at | Hs.82240 | 6809 | BE966922 | NM_004177 syntaxin 3A |
| 41 | 209268_at | Hs.6650 | 11311 | AF165513.1 | NM_007258 NM_007259 vacuolar |
| | | | | | protein sorting 45A NM_007259 |
| | | | | | vacuolar protein sorting 45A |
| 42 | 209295_at | Hs.51233 | 8795 | AF016266.1 | NM_003842 tumor necrosis factor |
| | | | | | receptor superfamily, member 10b |
| 43 | 209312_x_at | Hs.308026 | 3123 | U65585.1 | NM_002124 major histocompatibility |
| | | | | | complex, class II, DR beta 1 |
| | | | | | precursor |
| 44 | 209707_at | Hs.62187 | 10026 | AF022913.1 | |
| 45 | 209849_s_at | Hs.11393 | 5889 | AF029669.1 | NM_002876 RAD51 homolog C, |
| | | | | | isoform 2 NM_058216 RAD51 |
| | | | | | homolog C, isoform 1 NM_058217 |
| | | | | | RAD51 homolog C, isoform 3 |
| 46 | 209993_at | Hs.21330 | 5243 | AF016535.1 | NM_000927 ATP-binding cassette, |
| | | | | | sub-family B (MDR/TAP), member 1 |
| 47 | 210038_at | | | AL137145 | |
| 48 | 210039_s_at | Hs.211593 | 5588 | L01087.1 | NM_006257 protein kinase C, theta |
| 49 | 211061_s_at | Hs.172195 | 4247 | BC006390.1 | NM_002408 alpha-1,6-mannosyl- |
| | | | | | glycoprotein beta-1,2-N- |
| | | | | | acetylglucosaminyltransferase |
| 50 | 211769_x_at | Hs.272168 | 10955 | BC006088.1 | NM_006811 tumor differentially |
| | | | | | expressed 1 |
| 51 | 211833_s_at | Hs.159428 | 581 | U19599.1 | NM_004324 BCL2-associated X |
| | | | | | protein, isoform beta NM_138761 |
| | | | | | BCL2-associated X protein, isoform |
| | | | | | alpha NM_138762 BCL2-associated |
| | | | | | X protein, isoform gamma |
| | | | | | NM_138763 BCL2-associated X |
| | | | | | protein, isoform delta NM_138764 |
| | | | | | BCL2-associated X protein, isoform |
| | | | | | epsilon |
| 52 | 212120_at | Hs.250697 | 23433 | BF348067 | NM_012249 ras-like protein |
| 53 | 212266_s_at | Hs.166975 | 6430 | AW084582 | NM_006925 splicing factor, |
| | | | | | arginine/serine-rich 5 |
| 54 | 212359_s_at | Hs.65135 | 23053 | W89120 | |
| 55 | 212408_at | Hs.234265 | 26092 | AK023204.1 | |
| 56 | 212820_at | Hs.13264 | 23312 | AB020663.1 | NM_015263 rabconnectin-3 |
| 57 | 212998_x_at | Hs.73931 | 3119 | AI583173 | NM_002123 major histocompatibility |
| | | | | | complex, class II, DQ beta 1 |
| | | | | | precursor |
| 58 | 213056_at | Hs.96427 | 23150 | AU145019 | |
| 59 | 213258_at | Hs.288582 | | BF511231 | |
| 60 | 213400_s_at | Hs.76536 | 6907 | AV753028 | NM_005647 transducin beta-like 1X |
| 61 | 213624_at | Hs.42945 | 10924 | AA873600 | |
| 62 | 214098_at | Hs.21554 | 23285 | AB029030.1 | |
| 63 | 214449_s_at | Hs.250697 | 23433 | NM_012249.1 | NM_012249 ras-like protein |
| 64 | 215719_x_at | | | X83493.1 | |
| 65 | 215785_s_at | Hs.258503 | 26999 | AL161999.1 | |
| 66 | 216231_s_at | Hs.75415 | 567 | AW188940 | NM_004048 beta-2-microglobulin |
| 67 | 216860_s_at | Hs.339699 | 10220 | AF028333.1 | NM_005811 growth differentiation |
| | | | | | factor 11 |
| 68 | 217127_at | | | AL354872 | |
| 69 | 217731_s_at | Hs.239625 | 9445 | NM_021999.1 | NM_021999 integral membrane |
| | | | | | protein 2B |
| 70 | 217858_s_at | Hs.172788 | 51566 | NM_016607.1 | NM_016607 ALEX3 protein |
| 71 | 218007_s_at | Hs.108957 | 51065 | NM_015920.1 | NM_015920 ribosomal protein S27- |
| | | | | | like protein |
| 72 | 218084_x_at | Hs.333418 | 53827 | NM_014164.2 | NM_014164 FXYD domain- |
| | | | | | containing ion transport regulator 5 |
| 73 | 218107_at | Hs.289069 | 80232 | NM_025160.1 | NM_025160 hypothetical protein |
| | | | | | FLJ21016 |
| 74 | 218113_at | Hs.160417 | 23670 | NM_013390.1 | NM_013390 transmembrane protein 2 |
| 75 | 218132_s_at | Hs.15580 | 79042 | NM_024075.1 | NM_024075 LENG5 protein |
| 76 | 218248_at | Hs.19525 | 63901 | NM_022074.1 | NM_022074 hypothetical protein |
| | | | | | FLJ22794 |
| 77 | 218341_at | Hs.72531 | 79717 | NM_024664.1 | NM_024664 hypothetical protein |
| | | | | | FLJ11838 |
| 78 | 218603_at | Hs.6679 | 51696 | NM_016217.1 | NM_016217 hHDC for homolog of |
| | | | | | Drosophila headcase |
| 79 | 218634_at | Hs.268557 | 23612 | NM_012396.1 | NM_012396 pleckstrin homology-like |
| | | | | | domain, family A, member 3 |
| 80 | 218765_at | Hs.33724 | 51092 | NM_015996.1 | NM_015996 CGI-40 protein |
| 81 | 218773_s_at | Hs.279754 | 22921 | NM_012228.1 | NM_012228 pilin-like transcription |
| | | | | | factor NM_016064 |
| 82 | 218853_s_at | Hs.57549 | 56180 | NM_019556.1 | NM_019556 hypothetical protein |
| | | | | | dJ473B4 |
| 83 | 219236_at | Hs.235873 | 79957 | NM_024897.1 | NM_024897 hypothetical protein |
| | | | | | FLJ22672 |
| 84 | 219329_s_at | Hs.9527 | 51374 | NM_016085.1 | NM_016085 apoptosis related |
| | | | | | protein APR-3 NM_080592 apoptosis |
| | | | | | related protein APR-3 |
| 85 | 219694_at | Hs.91165 | 54491 | NM_019018.1 | NM_019018 hypothetical protein |
| | | | | | FLJ11127 |
| 86 | 219880_at | Hs.94037 | 64942 | NM_022907.1 | NM_022907 hypothetical protein |
| | | | | | FLJ23053 |
| 87 | 220261_s_at | Hs.5268 | 55146 | NM_018106.1 | NM_018106 hypothetical protein |
| | | | | | FLJ10479 |
| 88 | 220387_s_at | Hs.142245 | 11147 | NM_007071.1 | NM_007071 HERV-H LTR- |
| | | | | | associating 3 |
| 89 | 220520_s_at | Hs.163629 | 54830 | NM_017681.1 | NM_017681 hypothetical protein |
| | | | | | FLJ20130 |
| 90 | 221004_s_at | Hs.111577 | 81618 | NM_030926.1 | NM_030926 integral membrane |
| | | | | | protein 3 |
| 91 | 221449_s_at | Hs.23047 | 81533 | NM_030790.1 | NM_030790 hypothetical protein |
| | | | | | CDA08 |
| 92 | 221487_s_at | Hs.111680 | 2029 | AF157510.1 | NM_004436 endosulfine alpha |
| 93 | 221958_s_at | Hs.250746 | 79971 | AA775681 | NM_024911 hypothetical protein |
| | | | | | FLJ23091 |
| 94 | 222138_s_at | | | AF158978.1 | |
| 95 | 222209_s_at | | | AK000684.1 | |
| 96 | 55093_at | Hs.86392 | 54480 | AA534198 | |
| 97 | 56256_at | Hs.33724 | 51092 | AA150165 | NM_015996 CGI-40 protein |

TABLE 2

| | | | Locus | Ref Seq | |
|---|---|---|---|---|---|
| | Affimetrix | Unigene | Link | (NM_)/Gene | |
| Nr. | ID | ID | ID | Bank ID | Ref Seq ID + Annotation |
| 1 | 200659_s_at | Hs.75323 | 5245 | NM_002634.2 | NM_002634 prohibitin |
| 2 | 200685_at | Hs.11482 | 9295 | AU146237 | NM_004768 splicing factor, |
| | | | | | arginine/serine-rich 11 |
| 3 | 200691_s_at | Hs.3069 | 3313 | BC000478.1 | NM_004134 heat shock 70 kD |
| | | | | | protein 9B (mortalin-2) |
| 4 | 200692_s_at | Hs.3069 | 3313 | NM_004134.1 | NM_004134 heat shock 70 kD |
| | | | | | protein 9B (mortalin-2) |
| 5 | 200831_s_at | Hs.119597 | 6319 | AA678241 | NM_005063 stearoyl-CoA |
| | | | | | desaturase (delta-9-desaturase) |
| 6 | 200884_at | Hs.173724 | 1152 | NM_001823.1 | NM_001823 creatine kinase, |
| | | | | | brain |
| 7 | 200987_x_at | Hs.152978 | 10197 | AA758755 | NM_005789 proteasome |
| | | | | | (prosome, macropain) activator |
| | | | | | subunit 3 (PA28 gamma; Ki) |
| 8 | 201013_s_at | Hs.117950 | 10606 | AA902652 | NM_006452 |
| | | | | | phosphoribosylaminoimidazole |
| | | | | | carboxylase, |
| | | | | | phosphoribosylaminoribosylaminoimidazo |
| 9 | 201016_at | Hs.4310 | 1964 | BE542684 | NM_001412 eukaryotic |
| | | | | | translation initiation factor 4C |
| 10 | 201017_at | Hs.362973 | | BE542684 | |
| 11 | 201231_s_at | Hs.254105 | 2023 | NM_001428.1 | NM_001428 enolase 1 |
| | | | | | NM_005945 |
| 12 | 201284_s_at | Hs.78223 | 327 | NM_001640.2 | NM_001640 N-acylaminoacyl- |
| | | | | | peptide hydrolase |
| 13 | 201376_s_at | Hs.808 | 3185 | AI591354 | NM_004966 heterogeneous |
| | | | | | nuclear ribonucleoprotein F |
| 14 | 201516_at | Hs.76244 | 6723 | NM_003132.1 | NM_003132 spermidine synthase |
| 15 | 201600_at | Hs.7771 | 11331 | NM_007273.1 | NM_007273 B-cell associated |
| | | | | | protein |
| 16 | 201625_s_at | Hs.56205 | 3638 | BE300521 | NM_005542 insulin induced gene 1 |
| 17 | 201797_s_at | Hs.159637 | 7407 | NM_006295.1 | NM_006295 valyl-tRNA |
| | | | | | synthetase 2 |
| 18 | 202115_s_at | Hs.134200 | 26155 | NM_015658.1 | NM_015658 DKFZP564C186 |
| | | | | | protein |
| 19 | 202147_s_at | Hs.7879 | 3475 | NM_001550.1 | NM_001550 interferon-related |
| | | | | | developmental regulator 1 |
| 20 | 202159_at | Hs.23111 | 2193 | NM_004461.1 | NM_004461 phenylalanine-tRNA |
| | | | | | synthetase-like protein |
| 21 | 202462_s_at | Hs.17585 | 9879 | NM_014829.1 | NM_014829 RNA helicase |
| | | | | | KIAA0801 NM_016130 |
| 22 | 203119_at | Hs.4253 | 79080 | NM_024098.1 | NM_024098 hypothetical protein |
| | | | | | MGC2574 |
| 23 | 203782_s_at | Hs.153880 | 5442 | NM_005035.1 | NM_005035 mitochondrial DNA- |
| | | | | | directed RNA polymerase |
| | | | | | precursor |
| 24 | 204004_at | Hs.176090 | 5074 | AI336206 | NM_002583 apoptosis response |
| | | | | | protein |
| 25 | 206158_s_at | Hs.2110 | 7555 | NM_003418.1 | NM_003418 zinc finger protein 9 |
| 26 | 206424_at | Hs.150595 | 1592 | NM_000783.1 | NM_000783 cytochrome P450, |
| | | | | | subfamily XXVIA, polypeptide 1, |
| | | | | | isoform 1 NM_057157 |
| | | | | | cytochrome P450, subfamily |
| | | | | | XXVIA, polypeptide 1, isoform 2 |
| 27 | 206445_s_at | Hs.20521 | 3276 | NM_001536.1 | NM_001536 HMT1 hnRNP |
| | | | | | methyltransferase-like 2 |
| 28 | 207001_x_at | Hs.75450 | 1831 | NM_004089.1 | NM_004089 delta sleep inducing |
| | | | | | peptide, immunoreactor |
| 29 | 207397_s_at | Hs.158309 | 3239 | NM_000523.1 | NM_000523 homeo box D13 |
| 30 | 207793_s_at | Hs.37427 | 2035 | NM_004437.1 | NM_004437 erythrocyte |
| | | | | | membrane protein band 4.1 |
| | | | | | (elliptocytosis 1, RH-linked) |
| 31 | 208152_s_at | Hs.169531 | 9188 | NM_004728.1 | NM_004728 DEAD/H (Asp-Glu- |
| | | | | | Ala-Asp/His) box polypeptide 21 |
| 32 | 208625_s_at | Hs.211568 | 1981 | AF104913.1 | NM_004953 eukaryotic |
| | | | | | translation initiation factor 4 |
| | | | | | gamma, 1 |
| 33 | 208763_s_at | Hs.75450 | 1831 | AL110191.1 | NM_004089 delta sleep inducing |
| | | | | | peptide, immunoreactor |
| 34 | 208910_s_at | Hs.78614 | 708 | L04636.1 | NM_001212 complement |
| | | | | | component 1, q subcomponent |
| | | | | | binding protein precursor |
| 35 | 208916_at | Hs.183556 | 6510 | AF105230.1 | NM_005628 solute carrier family |
| | | | | | 1 (neutral amino acid |
| | | | | | transporter), member 5 |
| 36 | 208920_at | Hs.300741 | 6717 | AV752215 | NM_003130 sorcin |
| 37 | 209218_at | Hs.71465 | 6713 | AF098865.1 | NM_003129 squalene |
| | | | | | monooxygenase |
| 38 | 210005_at | Hs.82285 | 2618 | D32051.1 | NM_000819 |
| | | | | | phosphoribosylglycinamide |
| | | | | | formyltransferase, |
| | | | | | phosphoribosylglycinamide |
| | | | | | synthetas |
| 39 | 210337_s_at | Hs.174140 | 47 | U18197.1 | NM_001096 ATP citrate lyase |
| 40 | 210347_s_at | Hs.130881 | 53335 | AF080216.1 | NM_018014 B-cell |
| | | | | | CLL/lymphoma 11A, isoform 2 |
| | | | | | NM_022893 B-cell |
| | | | | | CLL/lymphoma 11A, isoform 1 |
| | | | | | NM_138552 B-cell |
| | | | | | CLL/lymphoma 11A, isoform 4 |
| | | | | | NM_138553 B-cell |
| | | | | | CLL/lymphoma 11A, isoform 5 |
| | | | | | NM_138559 B-cell |
| | | | | | CLL/lymphoma 11A, isoform 3 |
| 41 | 211615_s_at | Hs.182490 | 10128 | M92439.1 | NM_133259 leucine-rich PPR- |
| | | | | | motif containing |
| 42 | 211708_s_at | Hs.119597 | 6319 | BC005807.1 | NM_005063 stearoyl-CoA |
| | | | | | desaturase (delta-9-desaturase) |
| 43 | 211929_at | Hs.249247 | 10151 | BE867771 | NM_005758 heterogeneous |
| | | | | | nuclear ribonucleoprotein A3 |
| 44 | 212009_s_at | Hs.75612 | 10963 | BF974063 | NM_006819 stress-induced- |
| | | | | | phosphoprotein 1 (Hsp70/Hsp90- |
| | | | | | organizing protein) |
| 45 | 212018_s_at | | | AK025446.1 | |
| 46 | 212174_at | Hs.171811 | 204 | AK023758.1 | NM_001625 adenylate kinase 2 |
| | | | | | isoform a NM_013411 adenylate |
| | | | | | kinase 2 isoform b |
| 47 | 212680_x_at | Hs.100623 | 26472 | BE305165 | NM_138689 phospholipase C, |
| | | | | | beta 3, neighbor |
| 48 | 213147_at | Hs.110637 | 3206 | NM_018951.1 | NM_018951 homeo box A10 |
| 49 | 213427_at | Hs.115823 | 10799 | NM_006638.1 | NM_006638 ribonuclease P, |
| | | | | | 40 kD subunit |
| 50 | 213581_at | Hs.351862 | | BF446180 | |
| 51 | 213664_at | Hs.91139 | 6505 | AW235061 | NM_004170 solute carrier family |
| | | | | | 1 (neuronal/epithelial high affinity |
| | | | | | glutamate transporter |
| 52 | 213892_s_at | Hs.28914 | 353 | AA927724 | NM_000485 adenine |
| | | | | | phosphoribosyltransferase |
| 53 | 214661_s_at | Hs.117487 | 8602 | R06783 | |
| 54 | 216212_s_at | | | AJ010395 | |
| 55 | 216397_s_at | | | AK024840.1 | |
| 56 | 217027_x_at | | | AC004941 | |
| 57 | 217408_at | | | AL050361.1 | |
| 58 | 217985_s_at | Hs.8858 | 11177 | AA102574 | NM_013448 bromodomain |
| | | | | | adjacent to zinc finger domain, |
| | | | | | 1A |
| 59 | 218081_at | Hs.274422 | 54976 | NM_017874.1 | NM_017874 hypothetical protein |
| | | | | | FLJ20550 |
| 60 | 218105_s_at | Hs.279652 | 51073 | NM_015956.1 | NM_015956 mitochondrial |
| | | | | | ribosomal protein L4 |
| 61 | 218112_at | Hs.157160 | 65993 | NM_023936.1 | NM_023936 mitochondrial |
| | | | | | ribosomal protein S34 |
| 62 | 218188_s_at | Hs.23410 | 26517 | NM_012458.1 | NM_012458 translocase of inner |
| | | | | | mitochondrial membrane 13 |
| | | | | | homolog |
| 63 | 218305_at | Hs.61790 | 79711 | NM_024658.1 | NM_024658 importin 4 |
| 64 | 218493_at | Hs.15277 | 79622 | NM_024571.1 | NM_024571 hypothetical protein |
| | | | | | FLJ22940 |
| 65 | 218680_x_at | Hs.300954 | 25764 | NM_016400.1 | NM_016400 Huntingtin |
| | | | | | interacting protein K |
| 66 | 218889_at | Hs.74899 | 64318 | NM_022451.1 | NM_022451 AD24 protein |
| 67 | 218893_at | Hs.103833 | 79763 | NM_024710.1 | NM_024710 hypothetical protein |
| | | | | | FLJ23469 |
| 68 | 219433_at | Hs.130732 | 54880 | NM_017745.1 | NM_017745 BCL-6 interacting |
| | | | | | corepressor, isoform 1 |
| | | | | | NM_020926 BCL-6 interacting |
| | | | | | corepressor, isoform, 2 |
| 69 | 221691_x_at | Hs.9614 | 4869 | AB042278.1 | NM_002520 nucleophosmin |
| | | | | | (nucleolar phosphoprotein B23, |
| | | | | | numatrin) |

1. A method for detecting teneurin signalling, which method comprises:
a) determining the presence of a cleaved teneurin product associated with teneurin signalling, wherein said cleaved teneurin product comprises at least a portion of the cytoplasmic domain of teneurin and targets to the cell nucleus; and
b) correlating the presence and/or amount of said cleaved teneurin product with teneurin signalling.

2. A method as claimed in claim 1, wherein said teneurin is teneurin-1, teneurin-2, teneurin-3 or teneurin-4.

3. A method as claimed in of the preceding claims wherein the cleaved teneurin product is formed in turnout cells.

4. A method as claimed in claim 1 or 2, wherein the cleaved teneurin product is formed in neurons.

5. A method as claimed in of the preceding claims further comprising providing a teneurin or a fragment thereof comprising at least a portion of the N terminal domain of teneurin and at least a portion of the C-terminal domain of teneurin, and a cellular component that cleaves teneurin.

6. A method as claimed in claim 5, wherein the teneurin is recombinant.

7. A method as claimed in claim 6 wherein the cleaved teneurin product comprises a tag or label.

8. A method as claimed in claim 7, wherein said determining step (b) comprises detecting said tag or label photometrically.

9. A method as claimed in claim 7, wherein said tag is selected from the group consisting of GFP, YFP, hemagluttanin, (Histidine)₇, a DNA binding domain.

10. A method as claimed in claim 9 wherein said determining step (b) comprises allowing said DNA binding domain to bind to a nucleic acid comprising regulatory sequences operably linked to a reporter gene and detecting activity of said reporter gene.

11. A method as claimed in claim 10 wherein said DNA binding domain comprises a GAL4 DNA binding domain.

12. A method as claimed in claim 9, wherein said tag is a DNA binding domain and further comprises an NFκB domain.

13. A method as claimed in the preceding claim wherein said determining step comprises determining the amount of said cleaved tenascin product.

14. A method as claimed in the preceding claims wherein the cleaved teneurin product regulates expression or activity of a cellular target.

15. A method as claimed in claim 14 further comprising detecting expression or activity of said cellular target.

16. A method as claimed in claim 15 wherein said cellular target is PAL.

17. A method as claimed in claim 15 wherein said cellular target is Zic.

18. A method as claimed in claim 15 wherein said cellular target is ponsin.

19. A method as claimed in claim 1, wherein the presence and/or amount of the cleaved teneurin product is correlated to a particular disease or condition.

20. A method as claimed in claim 19, wherein said disease or condition is dependent on cell proliferation and/or neuronal differentiation.

21. Use of a detectable cleaved teneurin product associated with teneurin signalling in a method of diagnosis of a neuropathology or cell pathology affected by teneurin signalling.

22. A method for assessing the ability of an agent to modulate teneurin signalling, comprising the steps of:
(a) contacting teneurin with at least one agent;
(b) detecting cleavage of said teneurin by a cellular component associated with teneurin signalling in the presence of said agent; and
(c) correlating a difference in cleavage of said teneurin relative to when said agent is absent with an indication of the presence of an agent effective in modulating teneurin signalling.

23. A method as claimed in claim 22 wherein step (a) is performed by perfusing a cell expressing recombinant teneurin with the agent.

24. A method for assessing the ability of an agent to modulate teneurin-mediated signalling, comprising the steps of:
(a) exposing a cell to an agent;
(b) detecting expression or activity of a gene regulated by teneurin in said cell; and
(c) correlating a change in expression or activity of said gene with the presence of a modulator of teneurin signalling.

25. The use of an agent detected by a method of claim 22 for the manufacture of a medicament for the treatment or prophylactic treatment of a neuropathological condition.

26. The use of an agent detected by a method of claim 22 for the manufacture of a medicament for the treatment or prophylactic treatment of tumourigenesis or cancer.

27. The use of a cleaved teneurin product associated with teneurin signalling, wherein said cleaved teneurin product comprises at least a portion of the cytoplasmic domain of teneurin and targets to the cell nucleus; for the manufacture of a medicament for the treatment or prophylactic treatment of tumourigenesis or cancer.

28. The use of a cleaved teneurin product associated with teneurin signalling, wherein said cleaved teneurin product comprises at least a portion of the cytoplasmic domain of teneurin and targets to the cell nucleus; for the manufacture of a medicament for the treatment or prophylactic treatment of a neuropathological condition.

29. A method of treating an individual in need of treatment or prophylactic treatment of tumorogenesis, cancer or a neuropathological condition, said method comprising administering an effective amount of an agent identified by claim 22 sufficient to ameliorate the symptoms of said individual.

30. A method treating an individual in need of treatment or prophylactic treatment of tumorogenesis, cancer or a neuropathological condition, said method comprising administering an effective amount of a cleaved teneurin product comprising at least a portion of the cytoplasmic domain of teneurin, which targets to the cell nucleus, sufficient to ameliorate the symptoms of said individual.

31. A composition comprising a cleaved teneurin product and a cellular target of the cleaved teneurin product.

32. The composition of claim 31, wherein said cleaved teneurin product comprises at least a portion of the cytoplasmic domain of teneurin and targets to the cell nucleus.

33. A composition as claimed in claim 31 wherein said cellular target is PML.

34. A composition as claimed in claim 31 wherein said cellular target is Zic.

35. A composition as claimed in claim 31 wherein said cellular target is ponsin.

36. A composition as claimed in claim 31 wherein said cellular target is myc.

37. A composition as claimed in claim 31 wherein said cellular target is p53.

38. A kit comprising a teneurin and a protease.
